(19) United States
(12) Patent Application Publication
(10) Pub. No.: US 2004/0018611 A1
(43) Pub. Date: Jan. 29, 2004

(54) Microfluidic devices for high gradient magnetic separation

(76) Inventors: Michael Dennis Ward, Los Alamos, NM (US); Piotr Grodzinski, Chandler, AZ (US); Robin Hui Liu, Chandler, AZ (US); Bradley Neal Engel, Chandler, AZ (US); Yingjie Liu, Chandler, AZ (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/202,462

(22) Filed: Jul. 23, 2002

(51) Int. Cl. C12M 1/34; G01N 33/553

(52) U.S. Cl. 435/287.2

(57) ABSTRACT

The present invention provides microfluidic devices that can be used to effect a number of manipulations on a sample to ultimately result in target analyte detection or quantification. The device provides at least one magnetic microchannel that is capable of separating magnetic or magnetically-labeled target analytes from non-magnetic materials. Further, a magnetic microchannel may sort materials according to their magnetic response. Alternatively, magnetic or magnetically-labeled components other than the target analytes can be retained by the magnetic microchannel and are thus removed from the target analytes. Depending on the specificity of the binding ligand, one can either separate a vast population of analytes sharing a common binding motif, or specifically retain a rare target analyte because of its recognition of a specific ligand on the magnetic particle.

### STATEMENT REGARDING FEDERALLY SPONSORED RESEARCH OR DEVELOPMENT

The invention resulted in part from work on U.S. Government contract 70NANB9H3012 and DARPA #MDA972-01-3-0001.

### FIELD OF THE INVENTION

The invention relates generally to methods and apparatus for conducting analyses, particularly microfluidic devices for the detection of target analytes.

### BACKGROUND OF THE INVENTION

Recent advances in molecular biology have provided the opportunity to identify pathogens, diagnose disease states, and perform forensic determinations by detecting a specific material in a sophisticated biological sample. In order to obtain higher sensitivity and reduce cost for such detections, there is a significant trend to reduce the sizes of the detection device. Thus, a number of microfluidic device have been developed, generally comprising a solid support with microchannels, utilizing a number of different wells, pumps, reaction chambers, and the like. EP 0637996 B1; EP 0637998 B1; WO96/39260; WO97/16835; WO98/13683; WO97/16561; WO97/43629; WO96/39252; WO96/15576; WO96/15450; WO97/37755; and WO97/27324; and U.S. Pat. Nos. 5,304,487; 5,071,531; 5,061,336; 5,747,169; 5,296,375; 5,110,745; 5,587,128; 5,498,392; 5,643,738; 5,750,015; 5,726,026; 5,35,358; 5,126,022; 5,770,029; 5,631,337; 5,569,364; 5,135,627; 5,632,876; 5,593,838; 5,585,069; 5,637,469; 5,486,335; 5,755,942; 5,681,484; and 5,603,351.

The quality and sensitivity of detections by these microfluidic devices depend on the amount of target analytes in a sample. When an analytes is rare in the sample, it is necessary and sometimes even critical to process the sample for the successful analysis and detection. Specifically, the target analytes may need to be concentrated, enriched, or purified from contaminants that will otherwise interfere with its analysis and detection. The paucity of efficient sample preparation and handling techniques remains a serious limitation for the routine use of microfluidic devices to analyze complex samples.

High gradient magnetic separation (HGMS) is a long established procedure for selectively retaining magnetic materials in a chamber or column disposed in a magnetic field. This technique has also been applied to non-magnetic targets, including biological materials, labeled with magnetic labels. The technique of HGMS is thoroughly discussed in U.S. Pat. Nos. 5,411,863 and 5,385,707. Briefly, a target analyte within a complex sample is labeled by a magnetic label through its association with a specific binding ligand that is conjugated to a coating on the particle. The target,analyte, thus coupled to a magnetic “label”, is suspended in a fluid which is then applied to the chamber. In the presence of a magnetic gradient supplied across the chamber, the magnetically labeled target analyte is retained in the chamber; materials which do not have magnetic labels pass through the chamber. The retained target analyte can then be eluted by changing the strength of, or by eliminating, the magnetic field. The selectivity for a desired target material is supplied by the specific binding ligand conjugated to the magnetic particle.

Frequently, the chamber for HGMS contains a matrix of magnetically susceptibility material such as a steel wool or wire matrix. When a magnetic field is applied across the chamber, a high magnetic field gradient will be locally induced within the chamber in volumes close to the surface of the matrix, permitting the retention of fairly weakly magnetized particles. These designs have several disadvantages. First, unwanted materials are often trapped in crevices of the magnetically susceptible materials; second, because the interstitial spaces within the device and from device to device are nonuniform, the result produced are quite variable. Accordingly, improvements were made by packing small uniform ferromagnetic beads in a column to generate uniform interstitial spaces, and coating these beads to limit non-specific binding and help seal spaces that might trap unwanted materials (U.S. Pat. Nos. 5,711,871; 5,705,059; 5,543,289). Although these improvements greatly increased the efficiency and repeatability of separations, the improved columns cannot be optimized for rare target separation. Magnetic field gradients and insterstitial channel size are fixed by the bead size chosen. Smaller beads will produce stronger gradients but also smaller channel sizes. Even with relatively large beads (300 μm), the resulting ˜30 μm channel size often requires pre-filtering, traps a significant amount of non-specific material and makes elution of target cells difficult.

It is an object in this invention to incorporate a miniaturized magnetic separation system in a microfluidic device for sample processing. It is also an object in the present invention to disclose a superior HGMS system that can produce a higher magnetic gradient and capture rare species in a sample as well as complexes that are weakly magnetized. It is yet another object of the present invention to provide a way of achieving efficient washing and sample processing and consequently a more sensitive and selective device for the detection of target analytes.

### SUMMARY OF THE INVENTION

In a first aspect, an embodiment of the present invention is a microfluidic device comprising a solid support. The solid support comprises a sample inlet port a sample outlet port, and at least one microchannel comprising at least one section with walls comprising magnetic beads and an inner diameter devoid of beads. In an embodiment, the magnetic beads are embedded in the walls. In another embodiment, the magnetic beads are coated onto the inner surface of the walls. In some embodiments, the microfluidic devices comprise a detection module. The detection module comprises a detection electrode, a self-assembled monolayer, a binding ligand, and a detection inlet port to receive a sample.

Another embodiment of the present invention is a microfluidic device comprising a solid support. The solid support comprises a sample inlet port, a sample outlet port, and at least one microchannel comprising a gradient inducing feature coated with a magnetic material. In an embodiment, a plurality of gradient inducing features are present. In an embodiment, the gradient-inducing feature is a sawtooth ridge. In another embodiment, the gradient inducing feature is a dome. In an embodiment, the magnetic material is an iron-nickel alloy.

In an embodiment, the present invention provides a microfluidic device comprising a solid support, where the solid support comprises a sample inlet port, at least one microchannel comprising at least one section filled with magnetic beads, a sample outlet port, and a detection module. The detection module includes a detection electrode, a self-assembled monolayer; a binding ligand; and a detection inlet port to receive a sample.

In another aspect, the present invention provides a method to process a target analyte in a sample. An embodiment includes providing a target analyte labeled with a magnetic label and introducing the analyte to a microfluidic device comprising a solid support. The solid support comprises a sample inlet port, at least one microchannel comprising at least one section with walls comprising magnetic beads, and a sample outlet port. The sample is introduced under conditions whereby the labeled target analyte binds to said walls. In some embodiments, other components of the sample are washed away, or the analyte may be treated.

In another embodiment, the present invention provides a method to process a target analyte in a sample. A target analyte labeled with a magnetic label is provided and introduced to a microfluidic device comprising a solid support comprising a sample inlet port, at least one microchannel comprising a gradient inducing feature coated with a magnetic material, and a sample outlet port. The sample is introduced under conditions whereby said labeled target analyte is transported toward said gradient inducing feature.

In another embodiment, the present invention provides a method to process a target analyte in a sample. Target analyte labeled with a magnetic label is provided and introduced to a microfluidic device comprising a solid support. The solid support comprises a sample inlet port, at least one microchannel comprising at least one section filled with magnetic beads, a sample outlet port, and a detection module. The detection module includes a detection electrode, a self-assembled monolayer, a binding ligand, and a detection inlet port to receive a sample. The sample is introduced under conditions whereby the target analyte binds to the channel.

### BRIEF DESCRIPTION OF THE DRAWINGS

FIG. 1 depicts one preferred embodiment of the present invention. The depicted microfluidic device comprises a solid support 100 that has a sample inlet port 20, a storage module 30, a labeling chamber 40, a magnetic microchannel 50, a sample outlet port 60, a waste outlet port 70, a releasing chamber 80, a waste storage module 90, and a detection module 105. The various components are in communication with their corresponding components through fluidic microchannels. The embodiment may additionally comprise cell handling modules, reaction modules, separation modules, valves, and pumps.

FIGS. 2-4 depict a number of preferred embodiments of magnetic microchannels. FIG. 2 depicts a magnetic microchannel 550 with magnetic beads 11 embedded on the outer surface of the channel. The embedded beads can be optionally non-uniform in size. FIG. 3 depicts a magnetic microchannel 551 with magnetic beads 11 coated on the inner surface of the channel. The coated beads can be optionally non-uniform in size. FIG. 4 depicts a magnetic microchannel 552 with magnetic beads 11 packed inside the channel.

FIG. 5 depicts a cross-sectional view of a magnetic microchannel incorporating saw-toothed ridges according to an embodiment of the present invention.

FIG. 6 depicts a cross-sectional view of a magnetic microchannel incorporating domed features according to another embodiment of the present invention.

FIG. 7 depicts a mold for fabricating a magnetic microchannel incorporating a dome structure according to an embodiment of the present invention.

FIG. 8 is a schematic representation of an anisotropic etched Si structure according to an embodiment of the present invention.

FIGS. 9 and 10 depict scanning electron microscope (SEM) images of an anisotropic etched Si structure used to mold a plastic substrate according to an embodiment of the present invention.

FIGS. 11 and 12 depict SEM images of a compression-molded plastic microchannel with ridge microstructures according to an embodiment of the present invention.

FIGS. 13 and 14 depict SEM images of pit structures of an isotropic etched Si stamper according to an embodiment of the present invention.

FIGS. 15 and 16 depict SEM images of a channel structure with micro-dome arrays obtained in a compression molding process according to an embodiment of the present invention.

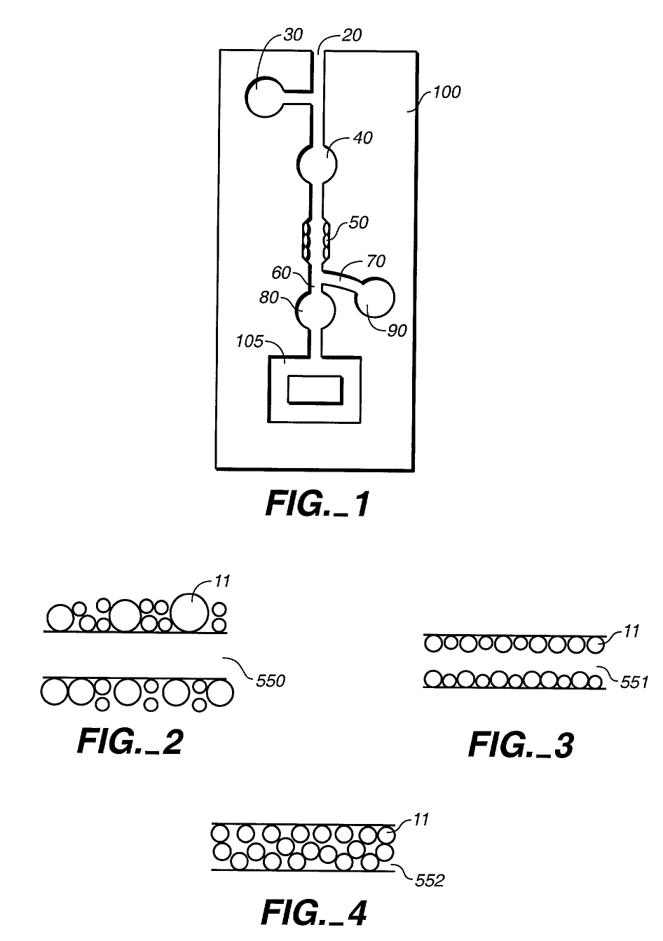

### DETAILED DESCRIPTION OF THE INVENTION

The present invention provides microfluidic devices that can be used to effect a number of manipulations on a sample to ultimately result in target analyte detection or quantification. The device provides at least one magnetic microchannel that is capable of separating magnetic or magnetically-labeled target analytes from non-magnetic materials. Further, a magnetic microchannel may sort materials according to their magnetic response. Alternatively, magnetic or magnetically-labeled components other than the target analytes can be retained by the magnetic microchannel and are thus removed from the target analytes. The magnetic labeling is achieved by the association of the target analyte or contaminant to a binding ligand conjugated on a magnetic particle. Depending on the specificity of the binding ligand, one can either separate a vast population of analytes sharing a common binding motif, or specifically retain a rare target analyte because of its recognition of a specific ligand on the magnetic particle.

The magnetic microchannel may comprise matrix elements such as magnetic beads that are either embedded in the substrate surrounding the microchannel or coated on the inner surface of the microchannel. Alternatively, the microchannel may be filled with magnetic beads, and the interstitial spacing among the beads form a relatively uniform channel in which the sample can flow. Upon being exposed to an external magnetic field, the magnetic beads will produce a local high gradient magnetic field within the microchannel. Advantageously, the particles that are embedded in or coated on the surfaces of the microchannels are nonuniform in size, so that a desired local magnetic gradient can be achieved.

In another preferred embodiment, the magnetic microchannel comprises a gradient-inducing feature. In this embodiment, one or more structural features are provided within the channel that enhance or induce a magnetic field gradient within magnetic microchannel. For example, in one embodiment a series of sawtooth ridges are provided, coated with a magnetic material. Gradient-inducing features are further described below.

In addition to the magnetic microchannels, there can also be other components integral to the microfluidic device. These include labeling chambers for attaching a magnetic label to a component in the sample; releasing chambers for releasing a magnetic label from the labeled component, cell handling modules for cell concentration, cell lysis, and cell removal; separation modules for separation of the desired target analyte from other sample components; and reaction modules for chemical or enzymatic reactions on the target analyte. The devices of the invention can also include one or more wells for sample manipulation, waste or reagents; microchannels to and between these wells; valves to control fluid movement; on-chip pumps; and detection modules for the detection of target analytes, as is more fully described below. The devices of the invention can be configured to manipulate one or multiple samples or analytes.

In an experiment, the biological sample is labeled with magnetic labels either in a separate device or within a labeling chamber integral to the microfluidic device. The labeled sample is then subjected to processing in a magnetic microchannel. Depending on the magnetic microchannels that are used, a magnetic field is generated within the channel either by an external magnet or by magnetizing magnetic materials within the channel. Materials that are labeled by magnetic labels will generally be retained in the magnetic microchannel, and those that are not captured in the channel can be collected for further processing or disposed as wastes. When target analytes are retained, they may be washed while captured within the microchannel. After the optional washing step, the target analytes can be directly detected within the magnetic microchannel, further processed in the microchannel, or eluted from the microchannel for further processing and/or detection. If processed inside the channel, the end product of the processing can also be eluted for further treatment and/or detection.

Accordingly, the present invention provides devices and methods for the detection of target analytes in biological samples. By “biological sample” herein is meant a sample containing at least one biological material. The list of biological materials includes but is not limited to microorganisms such as protozoa, bacteria, yeast, and other fungi, viruses, cultured cells or cells prepared from multi-cellular organisms including mammals and other vertebrates; bodily fluids including blood, lymph, saliva, vaginal and anal secretions, urine, feces, perspiration and tears; solid tissues, including liver, spleen, bone marrow, lung, muscle, brain, etc. Also appropriate are organelles or suborganelles of eucaryotic cells, and aggregates or individual molecules including proteins, glycoproteins, lipoproteins, carbohydrates, lipids, nucleic acids, and the like.

By “target analyte” or “analyte” or grammatical equivalents herein is meant any molecule, compound or particle to be detected. As outlined below, target analytes preferably bind to binding ligands, as is more fully described above. As will be appreciated by those in the art, a large number of analytes may be detected using the present methods; basically, any target analyte for which a binding ligand described herein, may be made may be detected using the methods of the invention.

Suitable analytes include organic and inorganic molecules, including biomolecules. In a preferred embodiment, the analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

In a preferred embodiment, the target analyte is a nucleic acid. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of electron transfer ligands, or to increase the stability and half-life of such molecules in physiological environments.

As will be appreciated by those in the art, all of these nucleic acid analogs may find use in the present invention. In addition, mixtures of naturally occurring nucleic acids and analogs can be made; for example, at the site of conductive oligomer or electron transfer ligand attachment, an analog structure may be used. Alternatively, mixtures of different nucleic acid analogs, and mixtures of naturally occurring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched base pairs. DNA and RNA typically exhibit a 2-4° C. drop in Tm for an internal mismatch. With the non-ionic PNA backbone, the drop is closer to 7-9° C. This allows for better detection of mismatches.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as nucleosides.

In a preferred embodiment, the present invention provides methods of detecting target nucleic acids. By “target nucleic acid” or “target sequence” or grammatical equivalents herein means a nucleic acid sequence on a single strand of nucleic acid. The target sequence may be a portion of a gene, a regulatory sequence, genomic DNA, cDNA, RNA including mRNA and rRNA, or others. It may be any length, with the understanding that longer sequences are more specific. In some embodiments, it may be desirable to fragment or cleave the sample nucleic acid into fragments of 100 to 10,000 base pairs, with fragments of roughly 500 base pairs being preferred in some embodiments. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others.

As is outlined more fully below, probes (including primers) are made to hybridize to target sequences to determine the presence or absence of the target sequence in a sample. Generally speaking, this term will be understood by those skilled in the art.

The target sequence may also be comprised of different target domains; for example, in “sandwich” type assays as outlined below, a first target domain of the sample target sequence may hybridize to a capture probe or a portion of capture extender probe, a second target domain may hybridize to a portion of an amplifier probe, a label probe, or a different capture or capture extender probe, etc. In addition, the target domains may be adjacent (i.e. contiguous) or separated. For example, when ligation chain reaction (LCR) techniques are used, a first primer may hybridize to a first target domain and a second primer may hybridize to a second target domain; either the domains are adjacent, or they may be separated by one or more nucleotides, coupled with the use of a polymerase and dNTPs, as is more fully outlined below.

The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

In a preferred embodiment, the target analyte is a protein. As will be appreciated by those in the art, there are a large number of possible proteinaceous target analytes that may be detected using the present invention. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration. As discussed below, when the protein is used as a binding ligand, it may be desirable to utilize protein analogs to retard degradation by sample contaminants.

Suitable protein target analytes include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C. perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lamblia Y. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphatase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone and testosterone; and (4) other proteins (including a-fetoprotein, carcinoembryonic antigen CEA, cancer markers, etc.).

In addition, any of the biomolecules for which antibodies may be detected may be detected directly as well; that is, detection of virus or bacterial cells, therapeutic and abused drugs, etc., may be done directly.

Suitable target analytes include carbohydrates, including but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242).

Particularly preferred target analytes include cells. “Cell” or “cells” as used herein refers to all types of cells, including prokaryotic and eukaryotic cells, such as bacterial, fungal, plant, and animal cells. In one embodiment the cells are plant cells, including both monocots and dicots and both angiosperms and gymnosperms, which cells may or may not include the cell wall. In another embodiment the cells are animal cells such as blood cells, including: end stage white blood cell types, such as neutrophils, eosinophils, basophils, T lymphocytes, B lymphocytes, macrophages and their monocyte antecedents; red blood cells and their reticulocyte antecedents; blood platelets and their megakaryocyte antecedents; intermediate forms; progenitor cells; and stem cells that give rise to all of these blood cells; other cells that may appear in the blood or other fluids from time to time such as blood vessel components, e.g. endothelial cells; fetal cells in pregnancy; and bacteria, protozoa and other parasites in blood.

The present invention provides microfluidic devices comprising solid supports. The “solid support” or “substrate” can be made of a wide variety of materials and can be configured in a large number of ways, as is discussed herein and will be apparent to one of skill in the art. In addition, a single device may comprise more than one substrate; for example, there may be a “sample processing” cassette that interfaces with a separate “detection” cassette; a raw sample is added to the sample processing cassette and is manipulated to prepare the sample for detection, which is removed from the sample processing cassette and added to the detection cassette. There may be an additional functional cassette into which the device fits; for example, a heating element which is placed in contact with the sample processing cassette to effect reactions such as PCR, or an electromagnet that produces a magnetic field across the chamber or magnetizes magnetic materials within the device. In some cases, a portion of the substrate may be removable; for example, the sample processing cassette may have a detachable detection cassette, such that the entire sample processing cassette is not contacted with the detection apparatus. See for example U.S. Pat. No. 5,603,351 and PCT US96/17116, hereby incorporated by reference.

The composition of the solid substrate will depend on a variety of factors, including the techniques used to create the device, the use of the device, the composition of the sample, the analyte to be detected, the size of the wells and microchannels, the presence or absence of electronic components, the choice of magnetic microchannels, etc. Generally, the devices of the invention should be easily sterilizable as well.

In a preferred embodiment, the solid substrate can be made from a wide variety of materials, including, but not limited to, silicon such as silicon wafers, silicon dioxide, silicon nitride, glass and fused silica, gallium arsenide, indium phosphide, aluminum, ceramics, polyimide, quartz, plastics, resins and polymers including polymethylmethacrylate, polydimethylsiloxane (PDMS), PMMA, epoxies, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, KOVAR, KEVLAR, KAPTON, MYLAR, brass, sapphire, etc. In preferred embodiments, the solid support is non-magnetic. In addition, as outlined herein, portions of the internal surfaces of the device may be coated with a variety of coatings as needed, to reduce non-specific binding, to generate a high gradient magnetic field, etc.

Materials that make up the magnetic microchannel are preferably nonmagnetic and generally may include any of the substrate materials listed above. Plastics, resins, polymers are preferred. PDMS, PMMA, polycarbonate, epoxies, and silicon wafers are particularly preferred. Non-magnetic metals such as aluminum or titanium are also suitable.

In some embodiments, where the target analytes are detected directly within the microchannel, certain optical requirements must also be met. One preferred mode of detection is light detection based for example on UV and visible, luminescence and fluorescence responses of the sample material to incident radiation. In this embodiment, any material used in fabricating the microchannel should have good optical transmittance, generally allowing at least about 50%, in some embodiments at least about 20%, and in still other embodiments at least about 10% transmittance. And, for example, any material that is to be used in the field of fluorescence detection and through which light passes should have sufficiently low fluorescence in the detected bandwidths so that background fluorescence does not interfere with detection of the signal from the sample material. Alternatively, as outlined below, electronic detection may be done, which negates the need for optical transparency.

The devices of the invention can be made in a variety of ways, as will be appreciated by those in the art. See for example WO96/39260, directed to the formation of fluid-tight electrical conduits; U.S. Pat. No. 5,747,169, directed to sealing; EP 0637996 B1; EP 0637998 B1; WO96/39260; WO97/16835; WO98/13683; WO97/16561; WO97/43629; WO96/39252; WO96/15576; WO96/15450; WO97/37755; and WO97/27324; and U.S. Pat. Nos. 5,304,487; 5,071531; 5,061,336; 5,747,169; 5,296,375; 5,110,745; 5,587,128; 5,498,392; 5,643,738; 5,750,015; 5,726,026; 5,35,358; 5,126,022; 5,770,029; 5,631,337; 5,569,364; 5,135,627; 5,632,876; 5,593,838; 5,585,069; 5,637,469; 5,486,335; 5,755,942; 5,681,484; and 5,603,351, all of which are hereby incorporated by reference. Suitable fabrication techniques again will depend on the choice of substrate, but preferred methods include, but are not limited to, a variety of micromachining and microfabrication techniques, including film deposition processes such as spin coating, chemical vapor deposition, laser fabrication, photolithographic and other etching techniques using either wet chemical processes or plasma processes, embossing, injection molding and bonding techniques (see U.S. Pat. No. 5,747,169, hereby incorporated by reference). In addition, there are printing techniques for the creation of desired fluid guiding pathways; that is, patterns of printed material can permit directional fluid transport. Thus, the build-up of “ink” can serve to define a flow channel. In addition, the use of different “inks” or “pastes” can allow different portions of the pathways having different flow properties.

For example, materials can be used to change solute/solvent RF values (the ratio of the distance moved by a particular solute to that moved by a solvent front). For example, printed fluid guiding pathways can be manufactured with a printed layer or layers comprised of two different materials, providing different rates of fluid transport. Multi-material fluid guiding pathways can be used when it is desirable to modify retention times of reagents in fluid guiding pathways. Furthermore, printed fluid guiding pathways can also provide regions containing reagent substances, by including the reagents in the “inks” or by a subsequent printing step. See for example U.S. Pat. No. 5,795,453, herein incorporated by reference in its entirety.

In a preferred embodiment, the solid substrate is configured for handling a single sample that may contain a plurality of target analytes. That is, a single sample is added to the device and the sample may either be aliquoted for parallel processing for detection of the analytes or the sample may be processed serially, with individual targets being detected in a serial fashion. In addition, samples may be removed periodically or from different locations for in line sampling.

In a preferred embodiment, the solid substrate is configured for handling multiple samples, each of which may contain one or more target analytes. In general, in this embodiment, each sample is handled individually; that is, the manipulations and analyses are done in parallel, with preferably no contact or contamination between them. Alternatively, there may be some steps in common; for example, it may be desirable to process different samples separately but detect all of the target analytes on a single detection electrode, as described below.

In addition, it should be understood that while most of the discussion herein is directed to the use of planar substrates with microchannels and wells, other geometries can be used as well. For example, two or more planar substrates can be stacked to produce a three dimensional device, that can contain microchannels flowing within one plane or between planes; similarly, wells may span two or more substrates to allow for larger sample volumes. Thus for example, both sides of a substrate can be etched to contain microchannels; see for example U.S. Pat. Nos. 5,603,351 and 5,681,484, both of which are hereby incorporated by reference.

Thus, the devices of the invention include at least one microchannel or flow channel that allows the flow of sample from the sample inlet port to the other components or modules of the system. The collection of microchannels and wells is sometimes referred to in the art as a “mesoscale flow system”. As will be appreciated by those in the art, the flow channels may be configured in a wide variety of ways, depending on the use of the channel. For example, a single flow channel starting at the sample inlet port may be separated into a variety of smaller channels, such that the original sample is divided into discrete subsamples for parallel processing or analysis. Alternatively, several flow channels from different modules, for example the sample inlet port and a reagent storage module may feed together into a mixing chamber or a reaction chamber. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer and sometimes tortuous flow channels can be used.

In general, the microfluidic devices of the invention are generally referred to as “mesoscale” devices. The devices herein are typically designed on a scale suitable to analyze microvolumes, although in some embodiments large samples (e.g. cc's of sample) may be reduced in the device to a small volume for subsequent analysis. That is, “mesoscale” as used herein refers to chambers and microchannels that have cross-sectional dimensions on the order of 0.1 μm to 500 μm. The mesoscale flow channels and wells have preferred depths on the order of 0.1 μm to 100 μm, typically 2-50 μm. The channels have preferred widths on the order of 2.0 to 500 μm, more preferably 3-100 μm. For many applications, channels of 5-50 μm are useful. However, for many applications, larger dimensions on the scale of millimeters may be used. Similarly, chambers (sometimes also referred to herein as “wells”) in the substrates often will have larger dimensions, on the scale of a few millimeters.

The microchannels may have any shape, for example, it may be linear, serpentine, arc shaped and the like. The cross-section of the channel may be circular, semicircular, ellipsoid, square, rectangular, trapezoidal, or other convenient configurations.

In a preferred embodiment, the microfluidic devices of the invention comprise at least one magnetic microchannel. By “magnetic microchannel” herein is meant microchannels that are capable of capturing and retaining magnetic or magnetically labeled materials, or sorting magnetic materials according to their magnetic response. As described below in more detail, the magnetic microchannel is capable of capturing magnetic or magnetically labeled materials because of the existence of a local high gradient magnetic field within the microchannel.

Generally, the magnetic microchannels are bigger than the fluid microchannels described above, and the exact dimension of the magnetic microchannels depends on the design of the magnetic microchannel, the desired magnetic field gradients, the size of the magnetic beads that make up the magnetic microchannel, and the chamber volume for reactions. Large gradients can be designed into a large or small channel. If the gradients are highly local, the channel may be made shallower to bring the analytes closer to the surface. A channel with both local and more global gradients, described further below, may have greater depth. Thus, the depth of the magnetic microchannel range from about 10 μm to 1 mm, usually from 50 μm to 500 μm, and most preferably from 100 μm to 300 μm. The width of the channel range from about 100 μm to 10 mm, more preferably 2 mm to 5 mm.

The length of the magnetic microchannels also depends on the residence time of the component to be captured. Some of the factors that are to be taken into consideration are concentration of the component, volume of starting materials, flow speed, channel width, gradient strength, and magnetic labeling efficiency. The preferred length of the magnetic microchannel range from 100 μm to 100 cm, more preferably from 500 μm to 50 mm, and most preferably from 1 mm to 30 mm.

When magnetic or magnetically labeled materials pass through the magnetic microchannel, they experience a magnetic force that draws them towards locations of high magnetic field strength. At the same time, these material also experience a shear force that tends to pull the material away. The materials will generally be captured when the magnetic force is greater than the shear force, with surface interactions between the channel and sample also sometimes influencing capture. The magnetic force that pulls the magnetic or magnetically labeled material depends on the magnetization of the material, as well as the local magnetic field gradient or the magnetic force density the material is exposed to. By “magnetization” herein is meant the magnetic moment per volume, typically measured in Bohr magnetons per unit volume. By magnetic field gradient hereby is meant a variation in the magnetic field with respect to a position. By magnetic force density herein is meant the magnetic force a particular particle encounters at its specific position. Gradients of about 10 T/m to 1000 T/m are generally appropriate for the separation of materials discussed herein, although in some cases a stronger or weaker gradient may be used.

In order to capture the magnetic or magnetically labeled materials, the time that takes the material to reach the surface of the channel or the matrix also has to be greater than the residence time of the material in the channel. The longer the distance from the initial location of the material to the channel wall or the surface of the matrix, the longer it takes the material to reach the wall. The residence time of the material in the magnetic microchannel depends on the flow rate of the sample. A slow flow rate will allow the magnetic or magnetically labeled material to stay longer in the magnetic microchannel, thus providing the material with more time and opportunity to be captured. The flow rate can be adjusted to balance capture efficiency with shear rate. A higher shear rate will generally result in cleaner separations but lower capture efficiency. The flow of the fluid may also be stopped temporarily if necessary.

In a preferred embodiment, the magnetic microchannel comprises magnetic beads. By “magnetic beads” herein is meant magnetically susceptible beads that are capable of producing high magnetic filed gradients in the channel when magnetized by an external magnetic field.

Materials for the magnetic beads include, but are not limited to, ferromagnetic, ferrimagnetic, or paramagnetic materials.

Ferromagnetism occurs when unpaired electrons in the material are contained in a crystalline lattice thus permitting coupling of the unpaired electrons. Ferromagnetic materials are strongly susceptible to magnetic fields and are capable of retaining magnetic properties when the field is removed. Preferred ferromagnetic materials include, but are not limited to, iron, cobalt, nickle, alloys thereof, and combinations thereof. Other ferromagnetic rare earth metals or alloys thereof are also suitable. The most preferred embodiment is nickel and alloys thereof because of its high chemical resistence and high magnetic permeability for very pure iron. In one embodiment, saw-tooth structures, described further below, were used and coated with a nickel-iron permalloy having a very high magnetic permeability.

In a preferred embodiment, the magnetic beads are very fine, typically about 10 to 500 μm. The relationship between the particle size and the magnetic force density produced by the particles in response to an external magnetic field is given by the equation

fₘ=B₀I grad H I=B₀M/a

where fₘ is magnetic force density, B₀ is the external magnetic field, I grad H I is the expression for the local gradient at the surface of a magnetic bead, M is the magnetization of the matrix element, and a is the diameter of the bead. Accordingly, the finer the magnetic beads, the higher the magnetic gradient and thus the higher a magnetic force density will be produced at the surface of the magnetic microchannel. Smaller beads will produce stronger gradients, but their effects will be more local. Generally, in a deeper channel only larger beads will produce gradients across the channel. This will allow the capture of very fine and weakly magnetized materials and increase the efficiency of magnetic capturing.

In a preferred embodiment, the magnetic beads are non-uniform in size. Generally, any shape beads may be used, that is, any shape having an angle or curvature will form gradients. Heterogeneous materials might be used to accomplish separations of targets with varying magnetic susceptibilities. While smaller magnetic beads produce higher magnetic force density, as explained above, larger beads produce a magnetic field gradient that reaches further from their surface. Generally, this is attributable to the higher radius of curvature of the smaller beads. Due to this smaller radius of curvature, smaller beads have stronger gradients at their surface than larger beads. The smaller beads also generally have gradients that fall off more rapidly with distance. Further, the magnetic flux at a distance will generally be less for a smaller bead. A mixture of small and big magnetic beads thus will capture both weakly magnetized materials (i.e., by smaller beads) and strongly magnetized materials that are far from the beads (i.e., by bigger beads). Combinations of magnetic beads with various sizes will allow one to create a desired gradient within the channel and create a high target capture efficiency. The present invention is in stark contrast to conventional magnetic separation techniques, which have emphasized on a uniform magnetic field inside the chamber/channel. In fact, in some applications of the present invention, uniform bead size is not necessarily a requirement and may even be detrimental to some applications. Preferred bead sizes generally range from about 10 μm-1 mm, although in some embodiments larger or smaller beads may be used.

In a preferred embodiment, the magnetic microchannel comprises at least one section comprising magnetic beads. This can be accomplished in three general ways: the magnetic beads may be embedded in one or more sections of the wall of the microchannel; the magnetic beads may be coated on one or more sections of the walls of the microchannel; or the magnetic beads may be packed into one or more sections of the microchannel.

By “walls” herein is meant the inner surface of the microchannel, or the substrate immediately surrounding the outer surface of the microchannel. By “section” herein is meant either a discrete area on the walls of the magnetic microchannel, or a portion of the inner channel chamber having the same diameter but a shorter length than the entire channel. Preferably, the wall or chamber along the entire length of the magnetic microchannel comprise magnetic beads for the highest efficiency. However, it is also possible that only one or more sections of the magnetic microchannel comprise magnetic beads.

The sections on the wall of the channel can have various sizes, shapes, and configurations. For example, one or more sections on the wall can be bands that surround the magnetic microchannel. Alternatively, the sections can be restricted to the lateral sides of the channel. The various sections can either be arranged in a variety of configurations, either randomly or in an ordered manner.

In a preferred embodiment, the magnetic beads are embedded in the walls of the magnetic microchannel (i.e., an “embedded channel”). More specifically, the magnetic beads are present in the substrate surrounding the outer surface of the microchannel. These beads can be in a single layer, or more preferably in multiple layers. The maximum number of the layers depends on the thickness of the substrate, the size of the beads, and/or the configuration of channels/components on the substrate.

While the magnetic beads in the embedded channel will generate a local high gradient magnetic field within the microchannel, they are present outside of the microchannel, thus guaranteeing a uniform flow within the channel and a consistent processing result. Because the sample flowing through the channel will not be in direct contact with the magnetic beads, many problems can be avoided. For example, avoiding direct contact between the magnetic beads with the samples eliminates the problem of non-specific binding or trapping of the sample in the channel, making it easier to wash and recover the sample. Damages to sensitive materials in the sample or to the magnetic beads due to direct contact between the sample and the magnetic beads can also be avoided. Furthermore, the channels can be easily washed after each experiment, making it possible to reuse the inventive device.

In a preferred embodiment, the magnetic beads are coated on the inner surface of the microchannel (i.e., a coated channel). Because the volume of the inner channel chamber will have to accommodate the magnetic beads, the depth of the microchannel will generally be deeper than the embedded channel. On the other hand, because the dimension of the microchannel is restricted by the overall design of the device, the number of the layers of beads in the coating could be limited. It is preferred, though not necessary, that the inner surface of the channel that is not coated with the magnetic beads is coated with a coating of the same thickness, so that the inner space of the channel will be uniform throughout the channel.

Like the embedded channel, the coated channel also allows a uniform flow within the channel and a consistent processing result. Furthermore, coated channels are easier to fabricate, and has less requirement for the material that makes up the channel, as described below.

In a preferred embodiment, the magnetic beads are packed into a microchannel (e.g., a “filled-channel”). The general design of a macroscale of such apparatus is taught by U.S. Pat. Nos. 5,705,059 and 5,711,871, incorporated herein as reference. Generally, the channel dimension is chosen according to the bead size in these embodiments. The design above requires uniform beads which would be in the range of about 10 μm-1 mm for a monolayer of beads. Devices could be designed with several layers, however, and the channel height may then be a multiple of this-generally, up to several millimeters for 1 mm beads.

In a preferred embodiment, the magnetic beads packed in the microchannel are substantially symmetrically spherical in shape. Such spheres can assume a lattice configuration wherein the gaps between the spheres form regular channels or pores in the matrix. The lattice configuration is a patterned framework of spheres that form channels of regular size between adjacent spheres and throughout the matrix. Upon the application of an external magnetic field to the magnetic microchannel, magnetic field gradients are created in the gaps between the spheres.

In a preferred embodiment, the sizes of the magnetic beads packed in the magnetic microchannel are relatively homogeneous, usually varying not more than about 15% from the average size, more usually by no more than 10%, and preferably by not more than about 5%. The uniform size, and therefore spacing, of the particles provides for a substantially uniform magnetic gradient throughout the matrix, and substantially uniform fluid flow characteristics.

In a preferred embodiment, the magnetic beads packed into the microchannel are coated with a materials as is generally described in U.S. Pat. No. 5,705,059. The coating materials include, but are not limited to, polymers such as plastic polymers, proteins, carbohydrates, organic molecules such as alkenes, etc. Coating is preferred in some embodiments because it helps to limit non-specific binding and to seal the spaces that might trap unwanted materials.

In a completed filled-channel, the selection of matrix and coating material will preferably result in channels or pathways through the matrix having an average diameter ranging from 1-100 μm and an occupying volume of about 60% to 80% of the total volume of the magnetic microchannel.

In a preferred embodiment, the magnetic beads in or on the walls of the magnetic microchannel are temporarily magnetic. For instance, they can be magnetized by an electromagnet and later demagnetized by reversing the polarity of the electromagnetic filed. By “electromagnet” herein is meant a mass, usually of soft iron, but sometimes of some other magnetic metal, such as nickel or cobalt, rendered temporarily magnetic by being placed within a coil of insulated wire through which a current of electricity is passing. The polarity of the electromagnet can be determined by controlling the direction of the electrical current in the wire. The electromagnet can be an integral part of the device, positioned at a convenient position proximal to the microchannel. Alternatively, the electromagnet can be a separate component from the device. The electromagnet should generally be positioned such that a field is produced perpendicular to the channel surface. The applied voltage can be any desired range to produce fields of about 0.1-1T.

In other preferred embodiments, the magnetic microchannel contains one or more gradient inducing features. By ‘gradient inducing’ feature herein is meant a physical feature that induces or enhances a magnetic gradient within the channel. Generally, any angled or curved feature will enhance or induce such a magnetic gradient. Accordingly, the gradient inducing feature may be a ridge, a sawtooth ridge, a dome, a step, a line, or any combination of these features. The slope and curvature of the gradient inducing feature is chosen based on the channel size, fabrication method, and desired gradient profile within the channel. In general, gradient inducing features of the present invention are between 1 μm and 1000 μm in height or diameter.

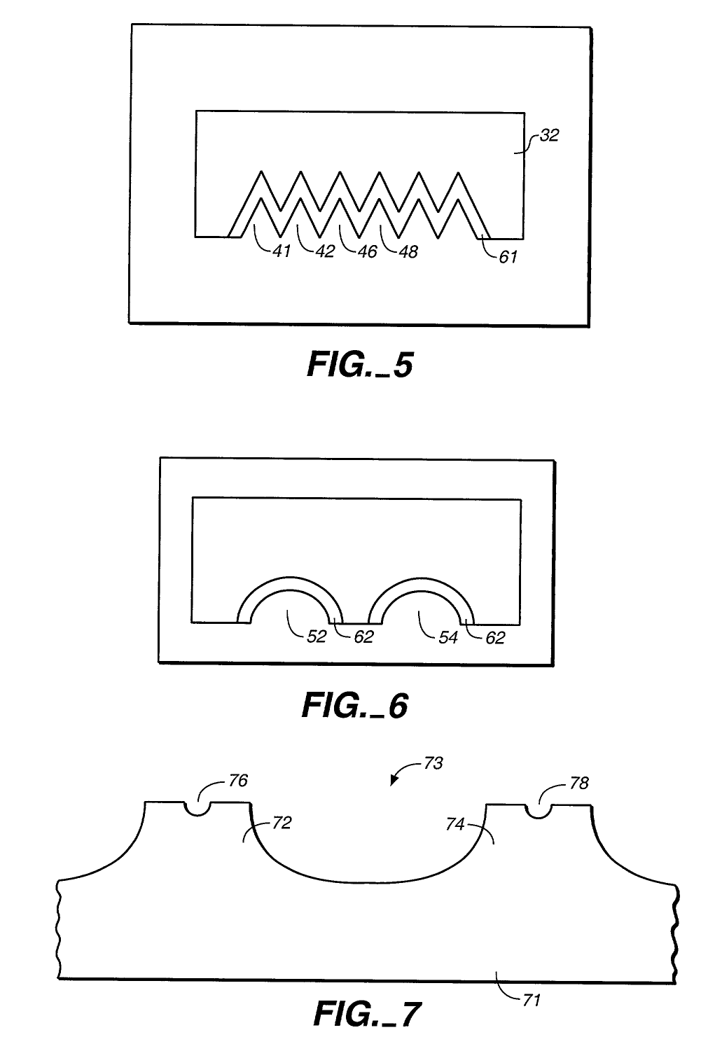

In a preferred embodiment, shown in FIG. 5, a cross-section of magnetic microchannel 32 is shown. It is noted that the device in FIG. 5 may be formed in a variety of ways, as described herein. A plurality of layers may be bonded or adhered together, for example, or in other embodiments the device may be injected molded. In still other embodiments sacrificial materials may be used to form microchannel 32. Although microchannel 32 is shown completely enclosed in FIG. 5, it is noted that all or any portion of magnetic microchannel 32 may be open. Magnetic microchannel 32 comprises a plurality of sawtooth ridges, including ridges 41, 42, 46, and 48 as shown in FIG. 5. In another preferred embodiment, shown in FIG. 6, the magnetic microchannel comprises an array of dome structures, such as domes 52 and 54. Although FIGS. 5 and 6 depict features (ridges or domes) along only one side of the microchannel, it is to be understood that gradient-inducing features may be fabricated on any side of the channel, and in some embodiments, gradient-inducing features are formed on two, three, four, or any other number of sides of the microchannel, as appropriate.

The gradient inducing feature is generally fabricated from the channel material, preferably plastic polymer, or acrylic, as discussed above, and coated with a magnetic material. The sawtooth ridges in FIG. 5 are coated with magnetic material 61. Although a continuous layer of magnetic material 60 is shown in FIG. 5, it is to be understood that magnetic material 61 may not be continuous in other embodiments. That is, magnetic material 61 may be formed, for example, only at the tips of ridges 41, 42, and 46 in FIG. 5. In a preferred embodiment, magnetic material 61 coating is a nickel-iron alloy comprising about 80 percent nickel and 20 percent iron. The magnetic material may also comprise any high magnetic permeability material that can be plated-iron, nickel, cobalt or alloys thereof, etc.

While FIGS. 5 and 6 show gradient inducing features only on one side of a microchannel, gradient inducing features may be placed on one or multiple sides of a channel. Further, gradient inducing features are preferably placed at an angle to the direction of fluid flow, particularly when the gradient inducing features are sharp features, such as sawtooth ridges. An area of low magnetic force may be present in the area between features. This effect is mediated by placing the features at an angle with respect to the fluid flow, or by filling the areas between features having low magnetic force with a non-magnetic material.

Gradient inducing features described above induce or enhance local useful magnetic field gradients, generally extending one half the diameter or height of the feature away from the feature. By ‘useful magnetic field gradient’ herein is meant a gradient of sufficient strength to influence an analyte of interest. Magnetic microchannels containing one or a plurality of gradient inducing features may be combined with one or more structures capable of generating a more global magnetic field gradient, that is a useful gradient that extends farther, in some embodiments up to distances on the order of millimeters. Suitable macro structures are described in U.S. Pat. Nos. 2,074,085; 6,241,894; and 6,013,188, all of which are expressly incorporated by reference herein.

In a preferred embodiment, the magnetic beads or magnetic materials coating gradient inducing features are permanently magnetized, for instance by a permanent magnet. Although less controllable, permanent magnet provides a cheaper and easier way of generating the magnetic field. The permanent magnet can either be an integral part of the device, or a separate component from the device. Preferably, the permanent magnet can be controlled by physically moving the magnet proximate or distal with respect to the magnetic microchannel.

Conveniently, the permanent magnet can be constructed of a commercially available alloy of neodinium/iron/boron. Other “off-the-shelf” magnets can also be used. Alternatively, the permanent magnet is carefully designed for the generation of an optimized magnetic filed, through the careful tuning of the key parameters such as magnetic material, geometry, configuration, and initial magnetization.

The use of external magnets to hold magnetically labeled components at a designated position in a microfluidic devices has been suggested previously in a number of patents, for example in U.S. Pat. Nos. 5,916,776, 5,939,291, and 6,193,892. Briefly, a magnetic field is generated by an external magnet, which will allow the immobilization of a material that is labeled with a magnetic label. In the present invention, the magnetic beads in the wall or the chamber of the microchannel will produce a local high magnetic field gradient upon the application of the external magnetic filed. The magnetic gradient produced by the magnetic beads will be 1 to 4 orders of magnitude greater than would be produced by the external magnet alone.

In a preferred embodiment, the microfluidic device comprises a magnetic labeling chamber for labeling the target analyte or any other component of the sample with magnetic labels. By “magnetic label” herein is meant magnetic particles conjugated with binding ligands to which the target analyte or other components of the sample can bind. In this embodiment, the reagent for the labeling reaction may contain the necessary reagents, or they may be stored in a storage module and pumped as needed. As will be appreciated by those skilled in the art, the labeling reaction described therein can also be carried out in a separate device.

By “magnetic particles” herein is meant magnetically susceptible particles that are small enough so that they can be manipulated in a microfluidic device. In a preferred embodiment, the labels are of any suitable shape, including rods and beads, and most preferably spherical beads. The labels have a preferred diameter of from about 0.01 μm to about 25 μm, more preferably, from about 0.05 μm to about 0.8 μm, yet more preferably from about 0.05 μm to about 0.2 μm.

In a preferred embodiment, the labels are ferromagnetic, paramagnetic, superparamagnetic, or made of any other material so that they can be seized or manipulated by a magnetic field within the magnetic microchannel. The material is preferably resistant to chemicals commonly used in manipulations of biological samples.

In a preferred embodiment, the magnetic particles are paramagnetic. “Paramagnetic” materials are characterized by containing unpaired electrons which are not coupled to each other through an organized matrix. They have only a weak magnetic susceptibility and when the field is removed quickly lose their weak magnetism. A paramagnetic particle can be comprised of, for example, iron dispersed in a polymer, and can be obtained, for example, from Miltenyi Biotec (Bergisch Gladbach, Germany or Immunicon (Huntingdon Valley, Pa.).

More preferably, the magnetic particles are superparamagnetic as sold by Dynal (Oslo, Norway) and other commercial manufacturers. Superparamagnetism occurs in ferromagnetic materials when the crystal diameter is decreased to less than a critical value. Superparamagnetic materials are highly magnetically susceptible-i.e., they become strongly magnetic when placed in a magnetic field, but, like paramagnetic materials, rapidly lose their magnetism. Whereas the paramagnetic particles exhibit some resonance and hysteresis, and therefore tend to clump together after exposure to a magnetic field ceases, superparamagnetic particles completely demagnetize when the field is removed, thus allowing the superparamagnetic particles to be redispersed without clumping after removal of the magnetic field.

Although the above-mentioned definitions are used for convenience, there is a continuum of properties between paramagnetic, superparamagnetic, and ferromagnetic, depending on crystal size and particle composition. Thus, these terms are used only for convenience, and “superparamagnetic” is intended to include a range of magnetic properties between the two designated extremes.

In a preferred embodiment, the magnetic particles are coated so that they can be conjugated to binding ligands that will enable them to capture the target analytes. Methods of conjugating a binding ligand to the magnetic particle are fully disclosed in U.S. Pat. Nos. 5,512,439 and 5,705,059, incorporated herein by reference. For conjugation purposes, a particularly preferred coating comprises of polymers or polysaccharide that either contain a functional group or are suitably derivatized to provide a functional group such as hydroxyl, carboxyl, sulfahydryl, aldehyde or amino groups. Such functional groups function to conjugate the coated particles to a specific binding ligand. A variety of suitable coatings are know to the art. For example, polyurethane together with a polyglycerol provides hydroxyl groups, a cellulose derivative provides a hydroxyl group, a polymer or copolymer of acrylic acid or methacrylic acid provide carboxyl groups, an aminoalkylated polymer provides amino groups. A variety of such modifications is known in the art. For example, polysaccharide can be conveniently oxidized using periodate to provide aldehyde functional groups which can then be conjugated to amino substituents on a proteinaceous binding ligand, or can be reacted with CNBr to provide this functionality.

By “binding ligands” or grammatical equivalents herein is meant a compound that can directly or indirectly bind to a component of the sample, which can either be a target analyte, or other analytes. In a preferred embodiment, the binding of the analytes to the binding ligand is specific, and the binding ligand is part of a binding pair. By “specifically bind” herein is meant that the ligand binds the component, for example the target analyte, with specificity sufficient to differentiate between the analyte and other components or contaminants of the test sample. The binding should be sufficient to remain bound under the conditions of the processing or treatment, including wash steps to remove non-specific binding. In some embodiments, the disassociation constants of the analyte to the binding ligand will be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred.

As will be appreciated by those in the art, the composition of the binding ligand will depend on the composition of the analyte to be labeled. Binding ligands to a wide variety of analytes are known or can be readily found using known techniques. As will be appreciated by those in the art, any two molecules that will associate, preferably specifically, may be used, either as the analyte or the binding ligand. Suitable analyte/binding ligand pairs include, but are not limited to, antibodies/antigens, receptors/ligand, proteins/nucleic acids; nucleic acids/nucleic acids, enzymes/substrates and/or inhibitors, carbohydrates (including glycoproteins and glycolipids)/lectins, carbohydrates and other binding partners, proteins/proteins; and protein/small molecules. These may be wild-type or derivative sequences.

In a preferred embodiment, the analyte is nucleic acid. The binding ligand for nucleic acids include sequence-specific binding ligands, as well as generic binding ligands. Sequence-specific binding ligands include, but is not limited to, a substantially complementary nucleic acid, or a sequence-specific nucleic-acid binding protein. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions. Generic binding ligands include, for example, single-stranded DNA binding proteins (SSB proteins), which can be expected to bind to all single-stranded DNA in a sample; poly-dT oligonucleotides, which can bind to substantially all the mRNA in the sample.

In a preferred embodiment, the analyte is protein. In this embodiment, the binding ligands include proteins, peptides, or small molecules. These binding ligands can be specific to a particular protein.

Alternatively, they may be recognizable by a particular class of proteins or even all proteins. For example, a specific binding ligand for a protein analyte can be specific antibodies or fragments thereof. When analyte is an enzyme, binding ligands can also be substrates, inhibitors, and other proteins that bind the enzyme, i.e. components of a multi-enzyme (or protein) complex. When target analyte is nucleic acid binding protein, the binding ligand can be a single-stranded or double-stranded nucleic acid.

In a preferred embodiment, the analyte is a cell. Binding ligands for a particular cell type generally comprise an antibody that recognize an epitope that serves to identify a particular cell type and distinguish it from other cell types. Suitable epitopes in this embodiment include, but are not limited to, components of the cell membrane, such as membrane-bound proteins or glycoproteins, including cell surface antigens of either host or viral origin, histocompatibility antigens or membrane receptors.

In a preferred embodiment, the binding ligands may be directly conjugated to the magnetic particles. Alternatively, the binding ligands and magnetic particles may be joined by means of a coupling agent. As used herein, coupling agents include various bifunctional cross-linking or coupling agents, i.e., molecules containing two reactive groups or ends, which may be separated by a spacer. The coupling agent contains both a binding ligand for the target analyte and a binding group for the molecule conjugated on the magnetic particle, thus brings the two together.

The method of attachment of the capture binding ligands to the attachment linker (either an insulator or conductive oligomer) will generally be done as is known in the art, and will depend on both the composition of the attachment linker and the capture binding ligand. In general, the capture binding ligands are attached to the attachment linker through the use of functional groups on each that can then be used for attachment. Preferred functional groups for attachment are amino groups, carboxy groups, oxo groups and thiol groups. These functional groups can then be attached, either directly or indirectly through the use of a linker, sometimes depicted herein as “Z”. Linkers are well known in the art; for example, homo-or hetero-bifunctional linkers as are well known (see 1994 Pierce Chemical Company catalog, technical section on cross-linkers, pages 155-200, incorporated herein by reference). Preferred Z linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C₂ alkene being especially preferred. Z may also be a sulfone group, forming sulfonamide linkages.

In a preferred embodiment, the coupling agent is a linker molecule. The linker can be an organic moiety such as a hydrocarbon chain (CH₂)ₙ, or can comprise an ether, ester, carboxyamide, or thioether moiety, or a combination thereof. The linker can also be an inorganic moiety such as siloxane (O—Si—O). The length of the linker is selected so that the magnetic particle does not interfere with the molecular interaction between the target analyte and its binding ligand.

In a preferred embodiment, the coupling agent comprises at least two parts, one part comprising a binding ligand for the analyte to be labeled, another part comprising an epitope that can be recognized by a binding ligand conjugated on the magnetic particle. This embodiment is particularly advantageous because a single kind of conjugated magnetic particle can be used for the labeling of a variety of target analytes. For example, Miltenyi Biotech strepavidin magnetic colloid labels can be used. These labels, together with a coupling agent comprising a biotinylated antibody can be used to label a cell or a protein that can be recognized by the biotinylated antibody. Similarly, the Miltenyi labels and a coupling agent comprising a biotinylated nucleic acid can be used to label a nucleic acid that is complementary to the biotinlyated nucleic acid.

Labeling reactions comprising more than one reaction step can be done in a variety of sequences. For example, the conjugated magnetic particles can first bind to the coupling agent, and the coupling agent/magnetic particle complex then reacts with the analyte in the sample. Alternatively, the coupling agent can first react with the analyte in the sample, and conjugated magnetic particles are subsequently introduced to the reaction. It is also possible that the analyte, the conjugated magnetic particle, and the coupling agent are allowed to bind to each other in a single reaction.

It should be noted that the labeled analytes may have various ratios of volume or numbers with regard to the labels. Thus, for large analytes such as cells, a multiplicity of labels may be attached to the cellular surface. On the other hand, if the analyte to be labeled is a single molecule, a multiplicity of such molecules may reside on a single label. Attaching a large nonmagnetic material, such as a cell to a magnetic particle alters the magnetic characteristics of the label to some extent due to the increased volume of the complex. Conversely, attaching a multiplicity of magnetic particles to a cell enhances the overall magnetization associated with the cell. The total magnetization of the labeled target in a magnetic field will thus depend on the individual magnetic moment of the particles, the size (volume) of the resulting labeled complex, and the number of magnetized particles per labeled complex.

In a preferred embodiment, more than one analytes in the sample are labeled in the labeling chamber. The different analytes can be labeled in a single labeling reaction, or, more preferably, in separate reactions or even separate labeling chambers.

In a preferred embodiment, the microfluidic device comprises a releasing chamber in which a target analyte that was attached to a magnetic label can be released from the label after being processed in the magnetic microchannel. The releasing chamber may contain the necessary reagents, or they may be stored in a storage module and pumped as needed.

In a preferred embodiment, the releasing reaction comprises a change in pH, salt concentration, temperature, etc.

In a preferred embodiment, the releasing reaction comprises an addition of competing ligands, detergents, chaotropic agents, organic compounds, or solvents, etc.

As will be appreciated by those in the art, the labeling chamber and the releasing chamber can be separate chambers that are dedicated to the labeling and releasing reactions. Alternatively, they can be part of the reaction module or other modules as described below. In addition, the releasing reaction described above can also be carried out in the magnetic microchannel.

As will be appreciated by those in the art and outlined below, the labeling chamber, the magnetic microchannel, and the releasing chamber can be integrated into the microfluidic devices of the invention in a wide variety of configurations. Specifically, a labeling chamber can be positioned anywhere before a magnetic microchannel, and a releasing chamber can be positioned anywhere in or after a magnetic microchannel.

In addition to the magnetic processing system, the devices of the invention are configured to include one or more of a variety of components, herein referred to as “modules”, that will be present on any given device depending on its use. These modules include, but are not limited to: sample inlet or outlet ports; sample introduction or collection modules; cell handling modules (for example, for cell lysis, cell removal, cell concentration, cell separation or capture, cell growth, etc.); separation modules, for example, for electrophoresis, dielectrophoresis, gel filtration, ion exchange/affinity chromatography etc.; reaction modules for chemical or biological alteration of the sample, including amplification of the target analyte (for example, when the target analyte is nucleic acid, amplification techniques are useful, including, but not limited to polymerase chain reaction (PCR), ligase chain reaction (LCR), strand displacement amplification (SDA), and nucleic acid sequence based amplification (NASBA)), chemical, physical or enzymatic cleavage or alteration of the target analyte, or chemical modification of the target; fluid pumps; fluid valves; thermal modules for heating and cooling; storage modules for assay reagents; mixing chambers; and detection modules.

In a preferred embodiment, the microfluidic devices of the invention comprise at least one sample inlet port for the introduction of the sample to the device. This may be part of or separate from a cell handling module, a reaction module, or a labeling chamber, that is, the sample may be directly fed in from the sample inlet port to the magnetic microchannel, or it may be pre-processed in other modules and transferred into the magnetic microchannel through a sample inlet port. Where there is only a single inlet, the inlet must serve to both admit samples to the magnetic microchannel and to admit solutions such as washing and elution solutions that pass through the magnetic channels. More preferably, one or more fluid inlets in addition to the sample inlet port are provided.

In a preferred embodiment, the microfluidic devices of the invention comprise at least one sample outlet port. By “sample outlet” port herein is meant the outlet port where the samples processed in the magnetic microchannel flow through. In addition, outlet ports for other microchannel of the invention are provided. The sample outlet port can be directly linked to a subsequent module (e.g., a reaction module, a separation module, or a detection module), or alternatively the sample can be collected from the outlet port and further processed. Where there is a single outlet port, the outlet port must serve both to discharge the flow-through portion of the sample that is not retained by the magnetic microchannel and to pass the portion that is bound to and subsequently eluted from the channel to subsequent processes. More preferably, there is at least one disposal outlet that is separate from the sample outlet port so that the flow-through sample can be disposed quickly without being mixed with the retained portion of the sample.

In a preferred embodiment, at least one sample outlet port or disposal outlet port is connected to a sample inlet port so that the samples can go through several rounds of processing either by the same magnetic microchannel or through additional channels in a multiple-channel arrangement in the same device or multiple devices. These multiple channels can either be of the same design or of various designs.

In a preferred embodiment, the devices of the invention include a sample collection module, which can be used to concentrate or enrich the sample if required; for example, see U.S. Pat. No. 5,770,029, including the discussion of enrichment channels and enrichment means.

In a preferred embodiment, the devices of the invention include a cell handling module. This is of particular use when the sample comprises cells that either contain the target analyte or that must be removed in order to detect the target analyte. Thus, for example, the detection of particular antibodies in blood can require the removal of the blood cells for efficient analysis, or the cells (and/or nucleus) must be lysed prior to detection. In this context, “cells” include eukaryotic and prokaryotic cells, and viral particles that may require treatment prior to analysis, such as the release of nucleic acid from a viral particle prior to detection of target sequences. In addition, cell handling modules may also utilize a downstream means for determining the presence or absence of cells. Suitable cell handling modules include, but are not limited to, cell lysis modules, cell removal modules, cell concentration modules, and cell separation or capture modules. In addition, as for all the modules of the invention, the cell handling module is in fluid communication via a flow channel with at least one other module of the invention.

In a preferred embodiment, the cell handling module includes a cell lysis module. Cells need to be lysed in order for the target analytes inside the cells to be magnetically labeled and processed in the magnetic microchannel. Alternatively, cells that have been separated by the magnetic microchannel need to be lysed before a target analyte within the cells can be detected.

As is known in the art, cells may be lysed in a variety of ways, depending on the cell type. In one embodiment, as described in EP 0 637 998 B1 and U.S. Pat. No. 5,635,358, hereby incorporated by reference, the cell lysis module may comprise cell membrane piercing protrusions that extend from a surface of the cell handling module. As fluid is forced through the device, the cells are ruptured. Similarly, this may be accomplished using sharp edged particles trapped within the cell handling region. Alternatively, the cell lysis module can comprise a region of restricted cross-sectional dimension, which results in cell lysis upon pressure.

In a preferred embodiment, the cell lysis module comprises a cell lysing agent, such as guanidium chloride, chaotropic salts, enzymes such as lysozymes, etc. In some embodiments, for example for blood cells, a simple dilution with water or buffer can result in hypotonic lysis. The lysis agent may be solution form, stored within the cell lysis module or in a storage module and pumped into the lysis module. Alternatively, the lysis agent may be in solid form, that is taken up in solution upon introduction of the sample.

The cell lysis module may also include, either internally or externally, a filtering module for the removal of cellular debris as needed. This filter may be microfabricated between the cell lysis module and the subsequent module to enable the removal of the lysed cell membrane and other cellular debris components; examples of suitable filters are shown in EP 0 637 998 B1, incorporated by reference.

In a preferred embodiment, the cell handling module includes a cell separation or capture module. This embodiment utilizes a cell capture region comprising binding sites capable of reversibly binding a cell surface molecule to enable the selective isolation (or removal) of a particular type of cell from the sample population, for example, white blood cells for the analysis of chromosomal nucleic acid, or subsets of white blood cells. These binding moieties may be immobilized either on the surface of the module or on a particle trapped within the module (i.e. a bead) by physical absorption or by covalent attachment. Suitable binding moieties will depend on the cell type to be isolated or removed, and generally includes antibodies and other binding ligands, such as ligands for cell surface receptors, etc. Thus, a particular cell type may be removed from a sample prior to further handling, or the assay is designed to specifically bind the desired cell type, wash away the non-desirable cell types, followed by either release of the bound cells by the addition of reagents or solvents, physical removal (i.e. higher flow rates or pressures), or even in situ lysis.

In a preferred embodiment, as described above, cell separation or capture can be achieved within the magnetic microchannel.

Alternatively, a cellular “sieve” can be used to separate cells on the basis of size. This can be done in a variety of ways, including protrusions from the surface that allow size exclusion, a series of narrowing channels, a weir, or a diafiltration type setup.

In a preferred embodiment, the cell handling module includes a cell removal module. This may be used when the sample contains cells that are not required in the assay or are undesirable. Generally, cell removal will be done on the basis of size exclusion as for “sieving”, above, with channels exiting the cell handling module that are too small for the cells.

In a preferred embodiment, the cell handling module includes a cell concentration module. As will be appreciated by those in the art, this is done using “sieving” methods, for example to concentrate the cells from a large volume of sample fluid prior to lysis.

In a preferred embodiment, the devices of the invention include a separation module. Separation in this context means that at least one component of the sample is separated from other components of the sample. Like the magnetic microchannel, the separation module can comprise the separation or isolation of the target analyte, or the removal of contaminants that interfere with the analysis of the target analyte, depending on the assay. The separation module may comprise one or more dielectrophoresis electrodes for separating sample components based on their dielectrophoretic response. Suitable separation modules for manipulating sample components via dielectrophoresis are described in U.S. Patent Application “Method and Apparatus for Manipulating Polarizable Analytes via Dielectrophoresis”, filed Jul. 22, 2002, incorporated herein by reference.

In a preferred embodiment, the separation module includes chromatographic-type separation media such as absorptive phase materials, including, but not limited to reverse phase materials (e.g. C₈ or C₁₈ coated particles, etc.), ion-exchange materials, affinity chromatography materials such as binding ligands, etc. See U.S. Pat. No. 5,770,029, herein incorporated by reference. Suitable choromatographic set ups for microfluidic devices include HPLC, CEC (capillary electrochromatography), as reviewed in Regnier et al., TIBTECH, March 1999, vol. 17.

In a preferred embodiment, the separation module utilizes binding ligands, as has been described above. When the sample component bound by the binding ligand is the target analyte, it may be released for detection purposes as described above.

In some embodiments, preferential binding of molecules to surfaces can be achieved using coating agents or buffer conditions; for example, DNA and RNA may be differentially bound to glass surfaces depending on the conditions.

In a preferred embodiment, the separation module includes an electrophoresis module, as is generally described in U.S. Pat. Nos. 5,770,029; 5,126,022; 5,631,337; 5,569,364; 5,750,015, and 5,135,627, all of which are hereby incorporated by reference. In electrophoresis, molecules are primarily separated by different electrophoretic mobilities caused by their different molecular size, shape and/or charge. Microcapillary tubes have recently been used for use in microcapillary gel electrophoresis (high performance capillary electrophoresis (HPCE)). One advantage of HPCE is that the heat resulting from the applied electric field is efficiently dissipated due to the high surface area, thus allowing fast separation. The electrophoresis module serves to separate sample components by the application of an electric field, with the movement of the sample components being due either to their charge or, depending on the surface chemistry of the microchannel, bulk fluid flow as a result of electroosmotic flow (EOF).

As will be appreciated by those in the art, the electrophoresis module can take on a variety of forms, and generally comprises an electrophoretic microchannel and associated electrodes to apply an electric field to the electrophoretic microchannel. Waste fluid outlets and fluid reservoirs are present as required.

The electrodes comprise pairs of electrodes, either a single pair, or, as described in U.S. Pat. Nos. 5,126,022 and 5,750,015, a plurality of pairs. Single pairs generally have one electrode at each end of the electrophoretic pathway. Multiple electrode pairs may be used to precisely control the movement of sample components, such that the sample components may be continuously subjected to a plurality of electric fields either simultaneously or sequentially.

In a preferred embodiment, electrophoretic gel media may also be used. By varying the pore size of the media, employing two or more gel media of different porosity, and/or providing a pore size gradient, separation of sample components can be maximized. Gel media for separation based on size are known, and include, but are not limited to, polyacrylamide and agarose. One preferred electrophoretic separation matrix is described in U.S. Pat. No. 5,135,627, hereby incorporated by reference, that describes the use of “mosaic matrix”, formed by polymerizing a dispersion of microdomains (“dispersoids”) and a polymeric matrix. This allows enhanced separation of target analytes, particularly nucleic acids. Similarly, U.S. Pat. No. 5,569,364, hereby incorporated by reference, describes separation media for electrophoresis comprising submicron to above-micron sized cross-linked gel particles that find use in microfluidic systems. U.S. Pat. No. 5,631,337, hereby incorporated by reference, describes the use of thermoreversible hydrogels comprising polyacrylamide backbones with N-substituents that serve to provide hydrogen bonding groups for improved electrophoretic separation. See also U.S. Pat. Nos. 5,061,336 and 5,071,531, directed to methods of casting gels in capillary tubes.

In a preferred embodiment, the devices of the invention include a reaction module. This can include either physical, chemical or biological alteration of one or more sample components. Alternatively, it may include a reaction module wherein the target analyte alters a second moiety that can then be detected; for example, if the target analyte is an enzyme, the reaction chamber may comprise an enzyme substrate that upon modification by the target analyte, can then be detected. In this embodiment, the reaction module may contain the necessary reagents, or they may be stored in a storage module and pumped as outlined herein to the reaction module as needed.

In a preferred embodiment, the reaction module includes a chamber for the chemical modification of all or part of the sample. For example, chemical cleavage of sample components (CNBr cleavage of proteins, etc.) or chemical cross-linking can be done. PCT US97/07880, hereby incorporated by reference, lists a large number of possible chemical reactions that can be done in the devices of the invention, including amide formation, acylation, alkylation, reductive amination, Mitsunobu, Diels Alder and Mannich reactions, Suzuki and Stille coupling, chemical labeling, etc. Similarly, U.S. Pat. Nos. 5,616,464 and 5,767,259 describe a variation of LCR that utilizes a “chemical ligation” of sorts. In this embodiment, similar to LCR, a pair of primers are utilized, wherein the first primer is substantially complementary to a first domain of the target and the second primer is substantially complementary to an adjacent second domain of the target (although, as for LCR, if a “gap” exists, a polymerase and dNTPs may be added to “fill in” the gap). Each primer has a portion that acts as a “side chain” that does not bind the target sequence and acts as one half of a stem structure that interacts non-covalently through hydrogen bonding, salt bridges, van der Waal's forces, etc. Preferred embodiments utilize substantially complementary nucleic acids as the side chains. Thus, upon hybridization of the primers to the target sequence, the side chains of the primers are brought into spatial proximity, and, if the side chains comprise nucleic acids as well, can also form side chain hybridization complexes. At least one of the side chains of the primers comprises an activatable cross-linking agent, generally covalently attached to the side chain, that upon activation, results in a chemical cross-link or chemical ligation. The activatible group may comprise any moiety that will allow cross-linking of the side chains, and include groups activated chemically, photonically and thermally, with photoactivatable groups being preferred. In some embodiments a single activatable group on one of the side chains is enough to result in cross-linking via interaction to a functional group on the other side chain; in alternate embodiments, activatable groups are required on each side chain. In addition, the reaction chamber may contain chemical moieties for the protection or deprotection of certain functional groups, such as thiols or amines.

In a preferred embodiment, the reaction module includes a chamber for the biological alteration of all or part of the sample. For example, enzymatic processes including nucleic acid amplification, hydrolysis of sample components or the hydrolysis of substrates by a target enzyme, the addition or removal of detectable labels, the addition or removal of phosphate groups, etc.

In a preferred embodiment, the target analyte is a nucleic acid and the biological reaction chamber allows amplification of the target nucleic acid. Suitable amplification techniques include, both target amplification and probe amplification, including, but not limited to, polymerase chain reaction (PCR), ligase chain reaction (LCR), strand displacement amplification (SDA), self-sustained sequence replication (3 SR), QB replicase amplification (QBR), repair chain reaction (RCR), cycling probe technology or reaction (CPT or CPR), and nucleic acid sequence based amplification (NASBA). Techniques utilizing these methods and the detection modules of the invention are described in PCT US99/01705, herein incorporated by reference in its entirety. In this embodiment, the reaction reagents generally comprise at least one enzyme (generally polymerase), primers, and nucleoside triphosphates as needed.

In a preferred embodiment when target analytes are amplified before being processed in the magnetic microchannel, the primers for the amplification reactions can be conjugated to a magnetic particle as described above. Thus the amplification products will be simultaneously labeled with magnetic labels and will be suitable for processing in the magnetic microchannel. Alternatively, ordinary, nonconjugated primers are used in an amplification reaction, and the amplified products will then be subjected to a subsequent labeling reaction prior to the processing in the magnetic microchannel.

General techniques for nucleic acid amplification are discussed below. In most cases, double stranded target nucleic acids are denatured to render them single stranded so as to permit hybridization of the primers and other probes of the invention. A preferred embodiment utilizes a thermal step, generally by raising the temperature of the reaction to about 95° C., although pH changes and other techniques such as the use of extra probes or nucleic acid binding proteins may also be used.

A probe nucleic acid (also referred to herein as a primer nucleic acid) is then contacted to the target sequence to form a hybridization complex. By “primer nucleic acid” herein is meant a probe nucleic acid that will hybridize to some portion, i.e. a domain, of the target sequence. Probes of the present invention are designed to be complementary to a target sequence (either the target sequence of the sample or to other probe sequences, as is described below), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the assays are generally run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The size of the primer nucleic acid may vary, as will be appreciated by those in the art, in general varying from 5 to 500 nucleotides in length, with primers of between 10 and 100 being preferred, between 15 and 50 being particularly preferred, and from 10 to 35 being especially preferred, depending on the use and amplification technique.

In addition, the different amplification techniques may have further requirements of the primers, as is more fully described below.

Once the hybridization complex between the primer and the target sequence has been formed, an enzyme, sometimes termed an “amplification enzyme”, is used to modify the primer. As for all the methods outlined herein, the enzymes may be added at any point during the assay, either prior to, during, or after the addition of the primers. The identification of the enzyme will depend on the amplification technique used, as is more fully outlined below. Similarly, the modification will depend on the amplification technique, as outlined below, although generally the first step of all the reactions herein is an extension of the primer, that is, nucleotides are added to the primer to extend its length.

Once the enzyme has modified the primer to form a modified primer, the hybridization complex is disassociated. Generally, the amplification steps are repeated for a period of time to allow a number of cycles, depending on the number of copies of the original target sequence and the sensitivity of detection, with cycles ranging from 1 to thousands, with from 10 to 100 cycles being preferred and from 20 to 50 cycles being especially preferred.

After a suitable time or amplification, the modified primer is moved to a detection module and incorporated into an assay complex, as is more fully outlined below. In some specific embodiments the assay complex is covalently attached to an electrode, and comprises at least one electron transfer moiety (ETM), described below. Electron transfer between the ETM and the electrode is then detected to indicate the presence or absence of the original target sequence, as described below. Alternatively, detection modules utilizing fluorescence are made, as described below.

In a preferred embodiment, the amplification is target amplification. Target amplification involves the amplification (replication) of the target sequence to be detected, such that the number of copies of the target sequence is increased. Suitable target amplification techniques include, but are not limited to, the polymerase chain reaction (PCR), strand displacement amplification (SDA), and nucleic acid sequence based amplification (NASBA).

In a preferred embodiment, the target amplification technique is PCR. The polymerase chain reaction (PCR) is widely used and described, and involve the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”. “panhandle PCR”, and “PCR select cDNA subtration”, among others.

In general, PCR may be briefly described as follows. A double stranded target nucleic acid is denatured, generally by raising the temperature, and then cooled in the presence of an excess of a PCR primer, which then hybridizes to the first target strand. A DNA polymerase then acts to extend the primer, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denaturation, annealing and extension. The particulars of PCR are well known, and include the use of a thermostabile polymerase such as Taq I polymerase and thermal cycling.

Accordingly, the PCR reaction requires at least one PCR primer and a polymerase. Mesoscale PCR devices are described in U.S. Pat. Nos. 5,498,392 and 5,587,128, and WO 97/16561, incorporated by reference.

In a preferred embodiment, the target amplification technique is SDA. Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby expressly incorporated by reference in their entirety.

In general, SDA may be described as follows. A single stranded target nucleic acid, usually a DNA target sequence, is contacted with an SDA primer. An “SDA primer” generally has a length of 25-100 nucleotides, with SDA primers of approximately 35 nucleotides being preferred. An SDA primer is substantially complementary to a region at the 3′ end of the target sequence, and the primer has a sequence at its 5′ end (outside of the region that is complementary to the target) that is a recognition sequence for a restriction endonuclease, sometimes referred to herein as a “nicking enzyme” or a “nicking endonuclease”, as outlined below. The SDA primer then hybridizes to the target sequence. The SDA reaction mixture also contains a polymerase (an “SDA polymerase”, as outlined below) and a mixture of all four deoxynucleoside-triphosphates (also called deoxynucleotides or dNTPs, i.e. dATP, dTTP, dCTP and dGTP), at least one species of which is a substituted or modified dNTP; thus, the SDA primer is modified, i.e. extended, to form a modified primer, sometimes referred to herein as a “newly synthesized strand”. The substituted dNTP is modified such that it will inhibit cleavage in the strand containing the substituted dNTP but will not inhibit cleavage on the other strand. Examples of suitable substituted dNTPs include, but are not limited, 2′deoxyadenosine 5′-O-(1-thiotriphosphate), 5-methyldeoxycytidine 5′-triphosphate, 2′-deoxyuridine 5′-triphosphate, and 7-deaza-2′-deoxyguanosine 5′-triphosphate. In addition, the substitution of the dNTP may occur after incorporation into a newly synthesized strand; for example, a methylase may be used to add methyl groups to the synthesized strand. In addition, if all the nucleotides are substituted, the polymerase may have 5′→3′ exonuclease activity. However, if less than all the nucleotides are substituted, the polymerase preferably lacks 5′→3′ exonuclease activity.

As will be appreciated by those in the art, the recognition site/endonuclease pair can be any of a wide variety of known combinations. The endonuclease is chosen to cleave a strand either at the recognition site, or either 3′ or 5′ to it, without cleaving the complementary sequence, either because the enzyme only cleaves one strand or because of the incorporation of the substituted nucleotides. Suitable recognition site/endonuclease pairs are well known in the art; suitable endonucleases include, but are not limited to, HincII, HindII, AvaI, Fnu4HI, TthIIII, NcII, BstXI, BamI, etc. A chart depicting suitable enzymes, and their corresponding recognition sites and the modified dNTP to use is found in U.S. Pat. No. 5,455,166, hereby expressly incorporated by reference.

Once nicked, a polymerase (an “SDA polymerase”) is used to extend the newly nicked strand, 5′→3′, thereby creating another newly synthesized strand. The polymerase chosen should be able to initiate 5′→3′ polymerization at a nick site, should also displace the polymerized strand downstream from the nick, and should lack 5′→3′ exonuclease activity (this may be additionally accomplished by the addition of a blocking agent). Thus, suitable polymerases in SDA include, but are not limited to, the Klenow fragment of DNA polymerase I, SEQUENASE 1.0 and SEQUENASE 2.0 (U.S. Biochemical), T5 DNA polymerase and Phi29 DNA polymerase.

Accordingly, the SDA reaction requires, in no particular order, an SDA primer, an SDA polymerase, a nicking endonuclease, and dNTPs, at least one species of which is modified.

In general, SDA does not require thermocycling. The temperature of the reaction is generally set to be high enough to prevent non-specific hybridization but low enough to allow specific hybridization; this is generally from about 37° C. to about 42° C., depending on the enzymes.

In a preferred embodiment, as for most of the amplification techniques described herein, a second amplification reaction can be done using the complementary target sequence, resulting in a substantial increase in amplification during a set period of time. That is, a second primer nucleic acid is hybridized to a second target sequence, that is substantially complementary to the first target sequence, to form a second hybridization complex. The addition of the enzyme, followed by disassociation of the second hybridization complex, results in the generation of a number of newly synthesized second strands.

In this way, a number of target molecules are made, and transferred to a detection module, described below. As is more fully outlined below, these reactions (that is, the products of these reactions) can be detected in a number of ways. In general, either direct or indirect detection of the target products can be done. “Direct” detection as used in this context, as for the other amplification strategies outlined herein, requires the incorporation of a label, in this case an electron transfer moiety (ETM), into the target sequence, with detection proceeding according to either “mechanism-1” or “mechanism-2”, outlined below. In this embodiment, the ETM(s) may be incorporated in three ways: (1) the primers comprise the ETM(s), for example attached to the base, a ribose, a phosphate, or to analogous structures in a nucleic acid analog; (2) modified nucleosides are used that are modified at either the base or the ribose (or to analogous structures in a nucleic acid analog) with the ETM(s); these ETM modified nucleosides are then converted to the triphosphate form and are incorporated into the newly synthesized strand by a polymerase; or (3) a “tail” of ETMs can be added, as outlined below. Either of these methods result in a newly synthesized strand that comprises ETMs, that can be directly detected as outlined below.

Alternatively, indirect detection proceeds as a sandwich assay, with the newly synthesized strands containing few or no ETMs. Detection then proceeds via the use of label probes that comprise the ETM(s); these label probes will hybridize either directly to the newly synthesized strand or to intermediate probes such as amplification probes, as is more fully outlined below. In this case, it is the ETMs on the label probes that are used for detection as outlined below.

In a preferred embodiment, the target amplification technique is nucleic acid sequence based amplification (NASBA). NASBA is generally described in U.S. Pat. No. 5,409,818 and “Profiting from Gene-based Diagnostics”, CTB International Publishing Inc., N.J., 1996, both of which are expressly incorporated by reference in their entirety.

In general, NASBA may be described as follows. A single stranded target nucleic acid, usually an RNA target sequence (sometimes referred to herein as “the first target sequence” or “the first template”), is contacted with a first NASBA primer. A “NASBA primer” generally has a length of 25-100 nucleotides, with NASBA primers of approximately 50-75 nucleotides being preferred. The first NASBA primer is preferably a DNA primer that has at its 3′ end a sequence that is substantially complementary to the 3′ end of the first template. The first NASBA primer has an RNA polymerase promoter at its 5′ end. The first NASBA primer is then hybridized to the first template to form a first hybridization complex. The NASBA reaction mixture also includes a reverse transcriptase enzyme (an “NASBA reverse transcriptase”) and a mixture of the four dNTPs, such that the first NASBA primer is modified, i.e. extended, to form a modified first primer, comprising a hybridization complex of RNA (the first template) and DNA (the newly synthesized strand).

By “reverse transcriptase” or “RNA-directed DNA polymerase” herein is meant an enzyme capable of synthesizing DNA from a DNA primer and an RNA template. Suitable RNA-directed DNA polymerases include, but are not limited to, avian myloblastosis virus reverse transcriptase (“AMV RT”) and the Moloney murine leukemia virus RT.

In addition to the components listed above, the NASBA reaction also includes an RNA degrading enzyme, also sometimes referred to herein as a ribonuclease, that will hydrolyze RNA of an RNA:DNA hybrid without hydrolyzing single- or double-stranded RNA or DNA. Suitable ribonucleases include, but are not limited to, RNase H from E. coli and calf thymus.

The ribonuclease degrades the first RNA template in the hybridization complex, resulting in a disassociation of the hybridization complex leaving a first single stranded newly synthesized DNA strand, sometimes referred to herein as “the second template”.

In addition, the NASBA reaction also includes a second NASBA primer, generally comprising DNA (although as for all the probes herein, including primers, nucleic acid analogs may also be used). This second NASBA primer has a sequence at its 3′ end that is substantially complementary to the 3′ end of the second template, and also contains an antisense sequence for a functional promoter and the antisense sequence of a transcription initiation site. Thus, this primer sequence, when used as a template for synthesis of the third DNA template, contains sufficient information to allow specific and efficient binding of an RNA polymerase and initiation of transcription at the desired site. Preferred embodiments utilizes the antisense promoter and transcription initiation site are that of the T7 RNA polymerase, although other RNA polymerase promoters and initiation sites can be used as well, as outlined below.

The second primer hybridizes to the second template, and a DNA polymerase, also termed a “DNA-directed DNA polymerase”, also present in the reaction, synthesizes a third template (a second newly synthesized DNA strand), resulting in second hybridization complex comprising two newly synthesized DNA strands.

Finally, the inclusion of an RNA polymerase and the required four ribonucleoside triphosphates (ribonucleotides or NTPs) results in the synthesis of an RNA strand (a third newly synthesized strand that is essentially the same as the first template). The RNA polymerase, sometimes referred to herein as a “DNA-directed RNA polymerase”, recognizes the promoter and specifically initiates RNA synthesis at the initiation site. In addition, the RNA polymerase preferably synthesizes several copies of RNA per DNA duplex. Preferred RNA polymerases include, but are not limited to, T7 RNA polymerase, and other bacteriophage RNA polymerases including those of phage T3, phage φII, Salmonella phage sp6, or Pseudomonase phage gh-1.

Accordingly, the NASBA reaction requires, in no particular order, a first NASBA primer, a second NASBA primer comprising an antisense sequence of an RNA polymerase promoter, an RNA polymerase that recognizes the promoter, a reverse transcriptase, a DNA polymerase, an RNA degrading enzyme, NTPs and dNTPs, in addition to the detection components outlined below.

These components result in a single starting RNA template generating a single DNA duplex; however, since this DNA duplex results in the creation of multiple RNA strands, which can then be used to initiate the reaction again, amplification proceeds rapidly.

As outlined herein, the detection of the newly synthesized strands can proceed in several ways. Direct detection can be done in the detection module when the newly synthesized strands comprise ETM labels, either by incorporation into the primers or by incorporation of modified labelled nucleotides into the growing strand. Alternatively, as is more fully outlined below, indirect detection of unlabelled strands (which now serve as “targets” in the detection mode) can occur using a variety of sandwich assay configurations. As will be appreciated by those in the art, it is preferable to detect DNA strands during NASBA since the presence of the ribonuclease makes the RNA strands potentially labile.

In a preferred embodiment, the amplification technique is signal amplification. Signal amplification involves the use of limited number of target molecules as templates to either generate multiple signaling probes or allow the use of multiple signaling probes. Signal amplification strategies include LCR, CPT, and the use of amplification probes in sandwich assays.

In a preferred embodiment, the signal amplification technique is LCR. The method can be run in two different ways; in a first embodiment, only one strand of a target sequence is used as a template for ligation; alternatively, both strands may be used. See generally U.S. Pat. Nos. 5,185,243 and 5,573,907; EP 0 320 308 B1; EP 0 336 731 B1; EP 0 439 182 B1; WO 90/01069; WO 89/12696; and WO 89/09835, and U.S. Ser. No. 60/078,102 and 60/073,011, all of which are incorporated by reference.

In a preferred embodiment, the single-stranded target sequence comprises a first target domain and a second target domain, and a first LCR primer and a second LCR primer nucleic acids are added, that are substantially complementary to its respective target domain and thus will hybridize to the target domains. These target domains may be directly adjacent, i.e. contiguous, or separated by a number of nucleotides. If they are non-contiguous, nucleotides are added along with means to join nucleotides, such as a polymerase, that will add the nucleotides to one of the primers. The two LCR primers are then covalently attached, for example using a ligase enzyme such as is known in the art. This forms a first hybridization complex comprising the ligated probe and the target sequence. This hybridization complex is then denatured (disassociated), and the process is repeated to generate a pool of ligated probes. In addition, it may be desirable to have the detection probes, described below, comprise a mismatch at the probe junction site, such that the detection probe cannot be used as a template for ligation.

In a preferred embodiment, LCR is done for two strands of a double-stranded target sequence. The target sequence is denatured, and two sets of probes are added: one set as outlined above for one strand of the target, and a separate set (i.e. third and fourth primer robe nucleic acids) for the other strand of the target. In a preferred embodiment, the first and third probes will hybridize, and the second and fourth probes will hybridize, such that amplification can occur. That is, when the first and second probes have been attached, the ligated probe can now be used as a template, in addition to the second target sequence, for the attachment of the third and fourth probes. Similarly, the ligated third and fourth probes will serve as a template for the attachment of the first and second probes, in addition to the first target strand. In this way, an exponential, rather than just a linear, amplification can occur.

Again, as outlined above, the detection of the LCR reaction can occur directly, in the case where one or both of the primers comprises at least one ETM, or indirectly, using sandwich assays, through the use of additional probes; that is, the ligated probes can serve as target sequences, and detection may utilize amplification probes, capture probes, capture extender probes, label probes, and label extender probes, etc.

In a preferred embodiment, the signal amplification technique is CPT. CPT technology is described in a number of patents and patent applications, including U.S. Pat. Nos. 5,011,769, 5,403,711, 5,660,988, and 4,876,187, and PCT published applications WO 95/05480, WO 95/1416, and WO 95/00667, and U.S. Ser. No. 09/014,304, all of which are expressly incorporated by reference in their entirety.

Generally, CPT may be described as follows. A CPT primer (also sometimes referred to herein as a “scissile primer”), comprises two probe sequences separated by a scissile linkage. The CPT primer is substantially complementary to the target sequence and thus will hybridize to it to form a hybridization complex. The scissile linkage is cleaved, without cleaving the target sequence, resulting in the two probe sequences being separated. The two probe sequences can thus be more easily disassociated from the target, and the reaction can be repeated any number of times. The cleaved primer is then detected as outlined herein.

By “scissile linkage” herein is meant a linkage within the scissile probe that can be cleaved when the probe is part of a hybridization complex, that is, when a double-stranded complex is formed. It is important that the scissile linkage cleave only the scissile probe and not the sequence to which it is hybridized (i.e. either the target sequence or a probe sequence), such that the target sequence may be reused in the reaction for amplification of the signal. As used herein, the scissile linkage, is any connecting chemical structure which joins two probe sequences and which is capable of being selectively cleaved without cleavage of either the probe sequences or the sequence to which the scissile probe is hybridized. The scissile linkage may be a single bond, or a multiple unit sequence. As will be appreciated by those in the art, a number of possible scissile linkages may be used.

In a preferred embodiment, the scissile linkage comprises RNA. This system, previously described in as outlined above, is based on the fact that certain double-stranded nucleases, particularly ribonucleases, will nick or excise RNA nucleosides from a RNA:DNA hybridization complex. Of particular use in this embodiment is RNAseH, Exo III, and reverse transcriptase.

In one embodiment, the entire scissile probe is made of RNA, the nicking is facilitated especially when carried out with a double-stranded ribonuclease, such as RNAseH or Exo III. RNA probes made entirely of RNA sequences are particularly useful because first, they can be more easily produced enzymatically, and second, they have more cleavage sites which are accessible to nicking or cleaving by a nicking agent, such as the ribonucleases. Thus, scissile probes made entirely of RNA do not rely on a scissile linkage since the scissile linkage is inherent in the probe.

In a preferred embodiment, when the scissile linkage is a nucleic acid such as RNA, the methods of the invention may be used to detect mismatches, as is generally described in U.S. Pat. Nos. 5,660,988, and WO 95/14106, hereby expressly incorporated by reference. These mismatch detection methods are based on the fact that RNAseH may not bind to and/or cleave an RNA:DNA duplex if there are mismatches present in the sequence. Thus, in the NA₁—R—NA₂ embodiments, NA₁ and NA₂ are non-RNA nucleic acids, preferably DNA. Preferably, the mismatch is within the RNA:DNA duplex, but in some embodiments the mismatch is present in an adjacent sequence very close to the desired sequence, close enough to affect the RNAseH (generally within one or two bases). Thus, in this embodiment, the nucleic acid scissile linkage is designed such that the sequence of the scissile linkage reflects the particular sequence to be detected, i.e. the area of the putative mismatch.

In some embodiments of mismatch detection, the rate of generation of the released fragments is such that the methods provide, essentially, a yes/no result, whereby the detection of the virtually any released fragment indicates the presence of the desired target sequence. Typically, however, when there is only a minimal mismatch (for example, a 1-, 2- or 3-base mismatch, or a 3-base delection), there is some generation of cleaved sequences even though the target sequence is not present. Thus, the rate of generation of cleaved fragments, and/or the final amount of cleaved fragments, is quantified to indicate the presence or absence of the target. In addition, the use of secondary and tertiary scissile probes may be particularly useful in this embodiment, as this can amplify the differences between a perfect match and a mismatch. These methods may be particularly useful in the determination of homozygotic or heterozygotic states of a patient.

In this embodiment, it is an important feature of the scissile linkage that its length is determined by the suspected difference between the target and the probe. In particular, this means that the scissile linkage must be of sufficient length to encompass the suspected difference, yet short enough the scissile linkage cannot inappropriately “specifically hybridize” to the selected nucleic acid molecule when the suspected difference is present; such inappropriate hybridization would permit excision and thus cleavage of scissile linkages even though the selected nucleic acid molecule was not fully complementary to the nucleic acid probe. Thus in a preferred embodiment, the scissile linkage is between 3 to 5 nucleotides in length, such that a suspected nucleotide difference from 1 nucleotide to 3 nucleotides is encompassed by the scissile linkage, and 0, 1 or 2 nucleotides are on either side of the difference.

Thus, when the scissile linkage is nucleic acid, preferred embodiments utilize from 1 to about 100 nucleotides, with from about 2 to about 20 being preferred and from about 5 to about 10 being particularly preferred.

CPT may be done enzymatically or chemically. That is, in addition to RNAseH, there are several other cleaving agents which may be useful in cleaving RNA (or other nucleic acid) scissile bonds. For example, several chemical nucleases have been reported; see for example Sigman et al., Annu. Rev. Biochem. 1990, 59, 207-236; Sigman et al., Chem. Rev. 1993, 93, 2295-2316; Bashkin et al., J. Org. Chem. 1990, 55, 5125-5132; and Sigman et al., Nucleic Acids and Molecular Biology, vol. 3, F. Eckstein and D. M. J. Lilley (Eds), Springer-Verlag, Heidelberg 1989, pp. 13-27; all of which are hereby expressly incorporated by reference.

Specific RNA hydrolysis is also an active area; see for example Chin, Acc. Chem. Res. 1991, 24, 145-152; Breslow et al., Tetrahedron, 1991, 47, 2365-2376; Anslyn et al., Angew. Chem. Int. Ed. Engl., 1997, 36, 432-450; and references therein, all of which are expressly incorporated by reference. Reactive phosphate centers are also of interest in developing scissile linkages, see Hendry et al., Prog. Inorg. Chem.: Bioinorganic Chem. 1990, 31, 201-258 also expressly incorporated by reference.

Current approaches to site-directed RNA hydrolysis include the conjugation of a reactive moiety capable of cleaving phosphodiester bonds to a recognition element capable of sequence-specifically hybridizing to RNA. In most cases, a metal complex is covalently attached to a DNA strand which forms a stable heteroduplex. Upon hybridization, a Lewis acid is placed in close proximity to the RNA backbone to effect hydrolysis; see Magda et al., J. Am. Chem. Soc. 1994, 116, 7439; Hall et al., Chem. Biology 1994, 1, 185-190; Bashkin et al., J. Am. Chem. Soc. 1994, 116, 5981-5982; Hall et al., Nucleic Acids Res. 1996, 24, 3522; Magda et al., J. Am. Chem. Soc. 1997, 119, 2293; and Magda et al., J. Am. Chem. Soc. 1997, 119, 6947, all of which are expressly incorporated by reference.

In a similar fashion, DNA-polyamine conjugates have been demonstrated to induce site-directed RNA strand scission; see for example, Yoshinari et al., J. Am. Chem. Soc. 1991, 113, 5899-5901; Endo et al., J. Org. Chem. 1997, 62, 846; and Barbier et al., J. Am. Chem. Soc. 1992, 114, 3511-3515, all of which are expressly incorporated by reference.

In a preferred embodiment, the scissile linkage is not necessarily RNA. For example, chemical cleavage moieties may be used to cleave basic sites in nucleic acids; see Belmont, et al., New J. Chem. 1997, 21, 47-54; and references therein, all of which are expressly incorporated herein by reference. Similarly, photocleavable moieties, for example, using transition metals, may be used; see Moucheron, et al., Inorg. Chem. 1997, 36, 584-592, hereby expressly by reference.

Other approaches rely on chemical moieties or enzymes; see for example Keck et al., Biochemistry 1995, 34, 12029-12037; Kirk et al., Chem. Commun. 1998, in press; cleavage of G-U base pairs by metal complexes; see Biochemistry, 1992, 31, 5423-5429; diamine complexes for cleavage of RNA; Komiyama, et al., J. Org. Chem. 1997, 62, 2155-2160; and Chow et al., Chem. Rev. 1997, 97, 1489-1513, and references therein, all of which are expressly incorporated herein by reference.

The first step of the CPT method requires hybridizing a primary scissile primer (also called a primary scissile probe) obe to the target. This is preferably done at a temperature that allows both the binding of the longer primary probe and disassociation of the shorter cleaved portions of the primary probe, as will be appreciated by those in the art. As outlined herein, this may be done in solution, or either the target or one or more of the scissile probes may be attached to a solid support. For example, it is possible to utilize “anchor probes” on a solid support or the electrode which are substantially complementary to a portion of the target sequence, preferably a sequence that is not the same sequence to which a scissile probe will bind.

Similarly, as outlined herein, a preferred embodiment has one or more of the scissile probes attached to a solid support such as a bead. In this embodiment, the soluble target diffuses to allow the formation of the hybridization complex between the soluble target sequence and the support-bound scissile probe. In this embodiment, it may be desirable to include additional scissile linkages in the scissile probes to allow the release of two or more probe sequences, such that more than one probe sequence per scissile probe may be detected, as is outlined below, in the interests of maximizing the signal.

In this embodiment (and in other techniques herein), preferred methods utilize cutting or shearing techniques to cut the nucleic acid sample containing the target sequence into a size that will allow sufficient diffusion of the target sequence to the surface of a bead. This may be accomplished by shearing the nucleic acid through mechanical forces or by cleaving the nucleic acid using restriction endonucleases. Alternatively, a fragment containing the target may be generated using polymerase, primers and the sample as a template, as in polymerase chain reaction (PCR). In addition, amplification of the target using PCR or LCR or related methods may also be done; this may be particularly useful when the target sequence is present in the sample at extremely low copy numbers. Similarly, numerous techniques are known in the art to increase the rate of mixing and hybridization including agitation, heating, techniques that increase the overall concentration such as precipitation, drying, dialysis, centrifugation, electrophoresis, magnetic bead concentration, etc.

In general, the scissile probes are introduced in a molar excess to their targets (including both the target sequence or other scissile probes, for example when secondary or tertiary scissile probes are used), with ratios of scissile probe:target of at least about 100:1 being preferred, at least about 1000:1 being particularly preferred, and at least about 10,000:1 being especially preferred. In some embodiments the excess of probe:target will be much greater. In addition, ratios such as these may be used for all the amplification techniques outlined herein.

Once the hybridization complex between the primary scissile probe and the target has been formed, the complex is subjected to cleavage conditions. As will be appreciated, this depends on the composition of the scissile probe; if it is RNA, RNAseH is introduced. It should be noted that under certain circumstances, such as is generally outlined in WO 95/00666 and WO 95/00667, hereby incorporated by reference, the use of a double-stranded binding agent such as RNAseH may allow the reaction to proceed even at temperatures above the Tm of the primary probe:target hybridization complex. Accordingly, the addition of scissile probe to the target can be done either first, and then the cleavage agent or cleavage conditions introduced, or the probes may be added in the presence of the cleavage agent or conditions.

The cleavage conditions result in the separation of the two (or more) probe sequences of the primary scissile probe. As a result, the shorter probe sequences will no longer remain hybridized to the target sequence, and thus the hybridization complex will disassociate, leaving the target sequence intact. The optimal temperature for carrying out the CPT reactions is generally from about 5° C. to about 25° C. below the melting temperatures of the probe:target hybridization complex. This provides for a rapid rate of hybridization and high degree of specificity for the target sequence. The Tm of any particular hybridization complex depends on salt concentration, G-C content, and length of the complex, as is known in the art.

During the reaction, as for the other amplification techniques herein, it may be necessary to suppress cleavage of the probe, as well as the target sequence, by nonspecific nucleases. Such nucleases are generally removed from the sample during the isolation of the DNA by heating or extraction procedures. A number of inhibitors of single-stranded nucleases such as vanadate, inhibitors it-ACE and RNAsin, a placental protein, do not affect the activity of RNAseH. This may not be necessary depending on the purity of the RNAseH and/or the target sample.

These steps are repeated by allowing the reaction to proceed for a period of time. The reaction is usually carried out for about 15 minutes to about 1 hour. Generally, each molecule of the target sequence will turnover between 100 and 1000 times in this period, depending on the length and sequence of the probe, the specific reaction conditions, and the cleavage method. For example, for each copy of the target sequence present in the test sample 100 to 1000 molecules will be cleaved by RNAseH. Higher levels of amplification can be obtained by allowing the reaction to proceed longer, or using secondary, tertiary, or quaternary probes, as is outlined herein.

Upon completion of the reaction, generally determined by time or amount of cleavage, the uncleaved scissile probes must be removed or neutralized prior to detection, such that the uncleaved probe does not bind to a detection probe, causing false positive signals. This may be done in a variety of ways, as is generally described below.

In a preferred embodiment, the separation is facilitated by the use of a solid support (either an internal surface of the device or beads trapped in the device) containing the primary probe. Thus, when the scissile probes are attached to the solid support, the flow of the sample past this solid support can result in the removal of the uncleaved probes.

In a preferred embodiment, the separation is based on gel electrophoresis of the reaction products to separate the longer uncleaved probe from the shorter cleaved probe sequences as is known in the art and described herein.

In a preferred embodiment, the separation is based on strong acid precipitation. This is useful to separate long (generally greater than 50 nucleotides) from smaller fragments (generally about 10 nucleotides). The introduction of a strong acid such as trichloroacetic acid into the solution (generally from a storage module) causes the longer probe to precipitate, while the smaller cleaved fragments remain in solution. The use of frits or filters can to remove the precipitate, and the cleaved probe sequences can be quantitated.

In a preferred embodiment, the scissile probe contains both an ETM and an affinity binding ligand or moiety, such that an affinity support is used to carry out the separation. In this embodiment, it is important that the ETM used for detection is not on the same probe sequence that contains the affinity moiety, such that removal of the uncleaved probe, and the cleaved probe containing the affinity moiety, does not remove all the detectable ETMs. Alternatively, the scissile probe may not contain a covalently attached ETM, but just an affinity label. Suitable affinity moieties include, but are not limited to, biotin, avidin, streptavidin, lectins, haptens, antibodies, etc. The binding partner of the affinity moiety is attached to a solid support (again, either an internal surface of the device or to beads trapped within the device) and the flow of the sample past this support is used to pull out the uncleaved probes, as is known in the art. The cleaved probe sequences, which do not contain the affinity moiety, remain insolution and then can be detected as outlined below.

In a preferred embodiment, similar to the above embodiment, a separation sequence of nucleic acid is included in the scissile probe, which is not cleaved during the reaction. A nucleic acid complementary to the separation sequence is attached to a solid support and serves as a catcher sequence. Preferably, the separation sequence is added to the scissile probes, and is not recognized by the target sequence, such that a generalized catcher sequence may be utilized in a variety of assays.

In a preferred embodiment, the uncleaved probe is neutralized by the addition of a substantially complementary neutralization nucleic acid, generally from a storage module. This is particularly useful in embodiments utilizing capture sequences, separation sequences, and one-step systems, as the complement to a probe containing capture sequences forms hybridization complexes that are more stable due to its length than the cleaved probe sequence:detection probe complex. As will be appreciated by those in the art, complete removal of the uncleaved probe is not required, since detection is based on electron transfer through nucleic acid; rather, what is important is that the uncleaved probe is not available for binding to a detection electrode probe specific for cleaved sequences. Thus, in one embodiment, this step occurs in the detection module and the neutralization nucleic acid is a detection probe on the surface of the electrode, at a separate “address”, such that the signal from the neutralization hybridization complex does not contribute to the signal of the cleaved fragments. Alternatively, the neutralization nucleic acid may be attached to a solid support; the sample flowed past the neutralization surface to quench the reaction, and thus do not enter the detection module.

After removal or neutralization of the uncleaved probe, detection proceeds via the addition of the cleaved probe sequences to the detection module, as outlined below, which can utilize either “mechanism-1” or “mechanism-2” systems.

In a preferred embodiment, no higher order probes are used, and detection is based on the probe sequence(s) of the primary primer. In a preferred embodiment, at least one, and preferably more, secondary probes (also referred to herein as secondary primers) are used. The secondary scissile probes may be added to the reaction in several ways. It is important that the secondary scissile probes be prevented from hybridizing to the uncleaved primary probes, as this results in the generation of false positive signal. In a preferred embodiment, the primary and secondary probes are bound to solid supports. It is only upon hybridization of the primary probes with the target, resulting in cleavage and release of primary probe sequences from the bead, that the now diffusible primary probe sequences may bind to the secondary probes. In turn, the primary probe sequences serve as targets for the secondary scissile probes, resulting in cleavage and release of secondary probe sequences. In an alternate embodiment, the complete reaction is done in solution. In this embodiment, the primary probes are added, the reaction is allowed to proceed for some period of time, and the uncleaved primary scissile probes are removed, as outlined above. The secondary probes are then added, and the reaction proceeds. The secondary uncleaved probes are then removed, and the cleaved sequences are detected as is generally outlined herein. In a preferred embodiment, at least one, and preferably more, tertiary probes are used. The tertiary scissile probes may be added to the reaction in several ways. It is important that the tertiary scissile probes be prevented from hybridizing to the uncleaved secondary probes, as this results in the generation of false positive signal. These methods are generally done as outlined above. Similarly, quaternary probes can be used as above.

Thus, CPT requires, again in no particular order, a first CPT primer comprising a first probe sequence, a scissile linkage and a second probe sequence; and a cleavage agent.

In this manner, CPT results in the generation of a large amount of cleaved primers, which then can be detected as outlined below.

In a preferred embodiment, the signal amplification technique is a “sandwich” assay, as is generally described in U.S. Ser. No. 60/073,011 and in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. Although sandwich assays do not result in the alteration of primers, sandwich assays can be considered signal amplification techniques since multiple signals (i.e. label probes) are bound to a single target, resulting in the amplification of the signal. Sandwich assays are used when the target sequence comprises little or no labels; that is, when a secondary probe, comprising the labels, is used to generate the signal.

As discussed herein, it should be noted that the sandwich assays can be used for the detection of primary target sequences (e.g. from a patient sample), or as a method to detect the product of an amplification reaction as outlined above; thus for example, any of the newly synthesized strands outlined above, for example using PCR, LCR, NASBA, SDA, etc., may be used as the “target sequence” in a sandwich assay.

Generally, sandwich signal amplification techniques may be described as follows. The reactions described below can occur either in the reaction module, with subsequent transfer to the detection module for detection, or in the detection module with the addition of the required components; for clarity, these are discussed together.

The methods include the addition of an amplifier probe, which is hybridized to the target sequence, either directly, or through the use of one or more label extender probes, which serves to allow “generic” amplifier probes to be made. Preferably, the amplifier probe contains a multiplicity of amplification sequences, although in some embodiments, as described below, the amplifier probe may contain only a single amplification sequence, or at least two amplification sequences. The amplifier probe may take on a number of different forms; either a branched conformation, a dendrimer conformation, or a linear “string” of amplification sequences. Label probes then hybridize to the amplification sequences (or in some cases the label probes hybridize directly to the target sequence), and the ETMs are detected, as is more fully outlined below.

As will be appreciated by those in the art, the systems of the invention may take on a large number of different configurations. In general, there are four types of systems that can be used: (1) “non-sandwich” systems (also referred to herein as “direct” detection) in which the target sequence itself is labeled (again, either because the primers comprise labels or due to the incorporation of labels into the newly synthesized strand); (2) systems in which label probes directly bind to the target analytes; (3) systems in which label probes are indirectly bound to the target sequences, for example through the use of amplifier probes; and (4) labelless electronic methods.

Detection of the amplification reactions of the invention, including the direct detection of amplification products, indirect detection utilizing label probes (i.e. sandwich assays) or detection of non-amplified targets, is done by detecting assay complexes comprising labels, which can be attached to the assay complex in a variety of ways, as is more fully described below.

In addition, as described in U.S. Pat. No. 5,587,128, the reaction chamber may comprise a composition, either in solution or adhered to the surface of the reaction chamber, that prevents the inhibition of an amplification reaction by the composition of the well. For example, the wall surfaces may be coated with a silane, for example using a silanization reagent such as dimethylchlorosilane, or coated with a siliconizing reagent such as Aquasil™ or Surfacil™ (Pierce, Rockford, Ill.), which are organosilanes containing a hydrolyzable group. This hydrolyzable group can hydrolyze in solution to form a silanol that can polymerize and form a tightly bonded film over the surface of the chamber. The coating may also include a blocking agent that can react with the film to further reduce inhibition; suitable blocking agents include amino acid polymers and polymers such as polyvinylpyrrolidone, proteins such as BSA, polyadenylic acid and polymaleimide. Alternatively, for silicon substrates, a silicon oxide film may be provided on the walls, or the reaction chamber can be coated with a relatively inert polymer such as a polyvinylchloride. In addition, it may be desirable to add blocking polynucleotides to occupy any binding sites on the surface of the chamber.

In a preferred embodiment, the biological reaction chamber allows enzymatic cleavage or alteration of the target analyte. For example, restriction endonucleases may be used to cleave target nucleic acids comprising target sequences, for example genomic DNA, into smaller fragments to facilitate either amplification or detection. Alternatively, when the target analyte is a protein, it may be cleaved by a protease. Other types of enzymatic hydrolysis may also be done, depending on the composition of the target analyte. In addition, as outlined herein, the target analyte may comprise an enzyme and the reaction chamber comprises a substrate that is then cleaved to form a detectable product.

In addition, in one embodiment the reaction module includes a chamber for the physical alteration of all or part of the sample, for example for shearing genomic or large nucleic acids, nuclear lysis, ultrasound, etc.

In a preferred embodiment, the above-mentioned reactions can be carried out within the magnetic microchannel while the magnetically labeled target analytes are still captured in the channel. Reaction reagents can be introduced into the magnetic microchannel either through a sample inlet port or from a separate fluid inlet port linked directly to the magnetic microchannel. In this embodiment, the magnetic microchannel is properly configured so that it can serve as a reaction chamber. For example, when a PCR reaction will be carried out inside the channel, it is necessary and sometimes essential that a thermal control module as described below is present underneath the channel.

In a preferred embodiments, a thermal module may be used, that is either part of the different modules or separate but can be brought into spatial proximity to the modules. The thermal module can include both heating and/or cooling capability. Suitable thermal modules are described in U.S. Pat. Nos. 5,498,392 and 5,587,128, and WO 97/16561, incorporated by reference, and may comprise electrical resistance heaters, pulsed lasers or other sources of electromagnetic energy directed to the reaction chamber. It should also be noted that when heating elements are used, it may be desirable to have the reaction chamber be relatively shallow, to facilitate heat transfer; see U.S. Pat. No. 5,587,128. Adequate thermal insulation surrounding the different modules to may also be desired to prevent unintended cross-heating among the modules. Temperature control is useful and sometimes essential for optimizing conditions for various chemical reactions in these modules, as well as binding and elution of target analytes in the magnetic microchannel.

In a preferred embodiment, the devices of the invention include at least one fluid pump. Pumps generally fall into two categories: “on chip” and “off chip”; that is, the pumps (generally electrode based pumps) can be contained within the device itself, or they can be contained on an apparatus into which the device fits, such that alignment occurs of the required flow channels to allow pumping of fluids.

In a preferred embodiment, the pumps are contained on the device itself. These pumps are generally electrode based pumps; that is, the application of electric fields can be used to move both charged particles and bulk solvent, depending on the composition of the sample and of the device. Suitable on chip pumps include, but are not limited to, electroosmotic (EO) pumps and electrohydrodynamic (EHD) pumps; these electrode based pumps have sometimes been referred to in the art as “electrokinetic (EK) pumps”. All of these pumps rely on configurations of electrodes placed along a flow channel to result in the pumping of the fluids comprising the sample components. As is described in the art, the configurations for each of these electrode based pumps are slightly different; for example, the effectiveness of an EHD pump depends on the spacing between the two electrodes, with the closer together they are, the smaller the voltage required to be applied to effect fluid flow. Alternatively, for EO pumps, the spacing between the electrodes should be larger, with up to one-half the length of the channel in which fluids are being moved, since the electrode are only involved in applying force, and not, as in EHD, in creating charges on which the force will act.

In a preferred embodiment, an electroosmotic pump is used. Electroosmosis (EO) is based on the fact that the surface of many solids, including quartz, glass and others, become variously charged, negatively or positively, in the presence of ionic materials. The charged surfaces will attract oppositely charged counterions in aqueous solutions. Applying a voltage results in a migration of the counterions to the oppositely charged electrode, and moves the bulk of the fluid as well. The volume flow rate is proportional to the current, and the volume flow generated in the fluid is also proportional to the applied voltage. Electroosmostic flow is useful for liquids having some conductivity is and generally not applicable for non-polar solvents. EO pumps are described in U.S. Pat. Nos. 4,908,112 and 5,632,876, PCT US95/14586 and WO97/43629, incorporated by reference.

In a preferred embodiment, an electrohydrodynamic (EHD) pump is used. In EHD, electrodes in contact with the fluid transfer charge when a voltage is applied. This charge transfer occurs either by transfer or removal of an electron to or from the fluid, such that liquid flow occurs in the direction from the charging electrode to the oppositely charged electrode. EHD pumps can be used to pump resistive fluids such as non-polar solvents. EHD pumps are described in U.S. Pat. No. 5,632,876, hereby incorporated by reference.

The electrodes of the pumps preferably have a diameter from about 25 microns to about 100 microns, more preferably from about 50 microns to about 75 microns. Preferably, the electrodes protrude from the top of a flow channel to a depth of from about 5% to about 95% of the depth of the channel, with from about 25% to about 50% being preferred. In addition, as described in PCT US95/14586, an electrode-based internal pumping system can be integrated into the liquid distribution system of the devices of the invention with flow-rate control at multiple pump sites and with fewer complex electronics if the pumps are operated by applying pulsed voltages across the electrodes; this gives the additional advantage of ease of integration into high density systems, reductions in the amount of electrolysis that occurs at electrodes, reductions in thermal convection near the electrodes, and the ability to use simpler drivers, and the ability to use both simple and complex pulse wave geometries.

The voltages required to be applied to the electrodes cause fluid flow depends on the geometry of the electrodes and the properties of the fluids to be moved. The flow rate of the fluids is a function of the amplitude of the applied voltage between electrode, the electrode geometry and the fluid properties, which can be easily determined for each fluid. Test voltages used may be up to about 1500 volts, but an operating voltage of about 40 to 300 volts is desirable. An analog driver is generally used to vary the voltage applied to the pump from a DC power source. A transfer function for each fluid is determined experimentally as that applied voltage that produces the desired flow or fluid pressure to the fluid being moved in the channel. However, an analog driver is generally required for each pump along the channel and is suitable an operational amplifier.

In a preferred embodiment, a micromechanical pump is used, either on- or off-chip, as is known in the art.

In a preferred embodiment, an “off-chip” pump is used. For example, the devices of the invention may fit into an apparatus or appliance that has a nesting site for holding the device, that can register the ports (i.e. sample inlet ports, fluid inlet ports, and waste outlet ports) and electrode leads. The apparatus can including pumps that can apply the sample to the device; for example, can force cell-containing samples into cell lysis modules containing protrusions, to cause cell lysis upon application of sufficient flow pressure. Such pumps are well known in the art.

In a preferred embodiment, the devices of the invention include at least one fluid valve that can control the flow of fluid into or out of a module of the device, or divert the flow into one or more channels. A variety of valves are known in the art. For example, in -one embodiment, the valve may comprise a capillary barrier, as generally described in PCT US97/07880, incorporated by reference. In this embodiment, the channel opens into a larger space designed to favor the formation of an energy minimizing liquid surface such as a meniscus at the opening. Preferably, capillary barriers include a dam that raises the vertical height of the channel immediate before the opening into a larger space such a chamber. In addition, as described in U.S. Pat. No. 5,858,195, incorporated herein by reference, a type of “virtual valve” can be used.

In a preferred embodiment, the devices of the invention include sealing ports, to allow the introduction of fluids, including samples, into any of the modules of the invention, with subsequent closure of the port to avoid the loss of the sample.

In a preferred embodiment, the devices of the invention include at least one storage modules for assay reagents. These are connected to other modules of the system using flow channels and may comprise wells or chambers, or extended flow channels. They may contain any number of reagents, buffers, enzymes, electronic mediators, salts, etc., including freeze dried reagents.

In a preferred embodiment, the devices of the invention include a mixing module; again, as for storage modules, these may be extended flow channels (particularly useful for timed mixing), wells or chambers. Particularly in the case of extended flow channels, there may be protrusions on the side of the channel to cause mixing.

In a preferred embodiment, the devices of the invention include a detection module. The detection module can be separate from the magnetic microchannel, or, more preferably, it is directly linked to the magnetic microchannel by at least one fluidic microchannel. Alternatively, it can be linked to any other modules of the devices of the invention.

In a preferred embodiment, the detection module comprises one or a multiplicity of arrays, particularly nucleic acid arrays, which are contained in one or a plurality of reaction volumes. By “array” or “biochip” herein is meant a plurality of capture binding ligands, preferably nucleic acids, in an array format; the size of the array will depend on the composition of the array. Most of the discussion therein is directed to the use of nucleic acid arrays with attached capture probes, but this is not meant to limit the scope of the invention, as other types of capture binding ligands (proteins, etc.) can be used. “Array” in this context generally refers to an ordered spacial arrangement, particularly an arrangement of immobilized biomolecules or polymeric anchoring structures. “Addressable array” refers to an array wherein the individual elements have precisely defined X and Y coordinates, so that a given element at a particular position in the array can be identified.

Nucleic acids arrays are known in the art, and can be classified in a number of ways; both ordered array (e.g. the ability to resolve chemistries at discrete sites), and random arrays are included. Ordered arrays include, but is not limited to, those made using photolithography techniques (Affimetrix GeneChip), spotting techniques (Synteni and others), printing techniques (Hewlett Packard and Rosetta), three dimensional “gel pad” arrays, electrochemical based electrode arrays, etc. The size of the array can vary, with arrays containing from about 2 different capture probes to many thousands can be made, with very large arrays being possible. Generally, depending on the type of array, the array will comprise from two to as many as 100,000, with from about 10 to about 1000 being the most preferred, and about 50 being especially preferred for electrode arrays. Arrays can also be classified as “addressable”, which means that the individual elements of the array have precisely defined coordinates, so that a given array element can be pinpointed.

In a preferred embodiment, the detection module is based on electrochemical or electronic methods and utilizes arrays of electrodes. In general, the detection module is based on work outlined in U.S. Pat. Nos. 5,591,578; 5,824,473; 5,770,369; 5,705,348 and 5,780,234; U.S. Ser. Nos. 09/096,593; 08/911,589; 09/135,183; and 60/105,875; and PCT applications US97/20014 and US98/12082; all of which are hereby incorporated by reference in their entirety. Detection module work is also outlined in WO98/20162, WO98/12430, WO00/16089, WO99/57317, WO01/35100, WO00/62931, WO01/06016, WO01/07665, WO01/54813, and WO01/42508; U.S. Pat. No. 6,232,062; and U.S. Ser. Nos. 09/459,685 and 09/458,533, all of which are hereby incorporated by reference.

There are two basic mechanisms which can be used in this embodiment. Both utilize detection electrodes with capture binding ligands attached (frequently referred to herein as “capture probes” when the analytes and ligands are nucleic acids). In one embodiment, detection is based on changes in impedance upon binding of the target analyte to the detection electrode. That is, the impedance between two electrodes is measured prior to the introduction of the sample comprising the target analyte, the analyte is introduced, the electrodes are washed if necessary, and then the impedance is measured again. This embodiment provides a significant commercial benefit, as no labels (e.g. electrochemical reporter molecules) are used, thus simplifying the reactions and costs of the system. These systems generally referred to herein as “impedance mode” systems and are generally described in WO98/20162, WO98/12430, WO00/16089, WO99/57317, WO01/35100, WO0/62931, WOO1/06016, WO01/07665, WO01/54813, and WO01/42508; U.S. Pat. No. 6,232,062; and U.S. Ser. Nos. 09/459,685 and 09/458,533, all of which are expressly incorporated by reference, and others of the above-listed applications.

Alternatively, electrochemical reporter groups (frequently referred to herein as electron transfer moieties (ETMs)) are used. In this embodiment, a target analyte is introduced to the detection module, and is combined with other components to form an assay complex in a variety of ways, as is more fully outlined below. The assay complexes comprise ETMs, which can be attached to the assay complex in a variety of ways, as is more fully described below. Detection proceeds by detecting the presence or absence of the ETMs as an indication of the presence or absence of the target analytes. These systems are generally referred to as “electron transfer mode” and are generally described in WO98/20162, WO98/12430, WO00/16089, WO99/57317, WO01/35100, WO00/62931, WO01/06016, WO01/07665, WO01/54813, and WO01/42508; U.S. Pat. No. 6,232,062; and U.S. Ser. Nos. 09/459,685 and 09/458,533, all of which are hereby incorporated by reference, all of which are expressly incorporated by reference, and others of the above-listed applications.

Accordingly, the detection modules of the invention comprise electrodes. By “electrode” herein is meant a composition, which, when connected to an electronic device, is able to sense a current or charge and convert it to a signal. Alternatively an electrode can be defined as a composition which can apply a potential to and/or pass electrons to or from species in the solution. Thus, an electrode is an ETM as described herein. Preferred electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂₀₆), tungsten oxide (WO₃) and ruthenium oxides; and carbon (including glassy carbon electrodes, graphite and carbon paste). Preferred electrodes include gold, silicon, platinum, carbon and metal oxide electrodes, with gold being particularly preferred.

In a preferred embodiment, the detection electrodes are formed on a substrate. In addition, the discussion herein is generally directed to the formation of gold electrodes, but as will be appreciated by those in the art, other electrodes can be used as well. The substrate can comprise a wide variety of materials, as will be appreciated by those in the art, with printed circuit board (PCB) materials being particularly preferred. Thus, in general, the suitable substrates include, but are not limited to, fiberglass, teflon, ceramics, glass, silicon, mica, plastic (including acrylics, polystyrene and copolymers of styrene and other materials, polypropylene, polyethylene, polybutylene, polycarbonate, polyurethanes, Teflon™, and derivatives thereof, etc.), GETEK (a blend of polypropylene oxide and fiberglass), etc.

In general, preferred materials include printed circuit board materials. Circuit board materials are those that comprise an insulating substrate that is coated with a conducting layer and processed using lithography techniques, particularly photolithography techniques, to form the patterns of electrodes and interconnects (sometimes referred to in the art as interconnections or leads). The insulating substrate is generally, but not always, a polymer. As is known in the art, one or a plurality of layers may be used, to make either “two dimensional” (e.g. all electrodes and interconnections in a plane) or “three dimensional” (wherein the electrodes are on one surface and the interconnects may go through the board to the other side) boards. Three dimensional systems frequently rely on the use of drilling or etching, followed by electroplating with a metal such as copper, such that the “through board” interconnections are made. Circuit board materials are often provided with a foil already attached to the substrate, such as a copper foil, with additional copper added as needed (for example for interconnections), for example by electroplating. The copper surface may then need to be roughened, for example through etching, to allow attachment of the adhesion layer.

Accordingly, in a preferred embodiment, the present invention provides biochips (sometimes referred to herein “chips”) that comprise substrates comprising a plurality of electrodes, preferably gold electrodes. The number of electrodes is as outlined for arrays. In “electron transfer mode”, preferably each electrode preferably comprises a self-assembled monolayer as outlined herein. In a preferred embodiment, one of the monolayer-forming species comprises a capture ligand as outlined herein. In addition, each electrode has an interconnection, that is attached to the electrode at one end and is ultimately attached to a device that can control the electrode. That is, each electrode is independently addressable.

The substrates can be part of a larger device comprising a detection chamber that exposes a given volume of sample to the detection electrode. Generally, the detection chamber ranges from about 1 nL to 1 ml, with about 10 μL to 500 μL being preferred. As will be appreciated by those in the art, depending on the experimental conditions and assay, smaller or larger volumes may be used.

In some embodiments, the detection chamber and electrode are part of a cartridge that can be placed into a device comprising electronic components (an AC/DC voltage source, an ammeter, a processor, a read-out display, temperature controller, light source, etc.). In this embodiment, the interconnections from each electrode are positioned such that upon insertion of the cartridge into the device, connections between the electrodes and the electronic components are established.

Detection electrodes on circuit board material (or other substrates) are generally prepared in a wide variety of ways and are described in the references outlined above.

The electrodes described herein are depicted as a flat surface, which is only one of the possible conformations of the electrode and is for schematic purposes only. The conformation of the electrode will vary with the detection method used. For example, flat planar electrodes may be preferred for optical detection methods, or when arrays of nucleic acids are made, thus requiring addressable locations for both synthesis and detection. Alternatively, for single probe analysis, the electrode may be in the form of a tube, with the SAMs comprising conductive oligomers and nucleic acids bound to the inner surface. Electrode coils may be preferred in some embodiments as well. This allows a maximum of surface area containing the nucleic acids to be exposed to a small volume of sample.

In “impedance mode” the detection electrode can comprise a coating of conductive polymers or oligomers. By “conductive oligomer” herein is meant a substantially conducting oligomer, preferably linear, some embodiments of which are referred to in the literature as “molecular wires”. By “substantially conducting” herein is meant that the oligomer is capable of transferring electrons at 100 Hz. Generally, the conductive oligomer has substantially overlapping n-orbitals, i.e. conjugated n-orbitals, as between the monomeric units of the conductive oligomer, although the conductive oligomer may also contain one or more sigma (σ) bonds. Additionally, a conductive oligomer may be defined functionally by its ability to inject or receive electrons into or from an associated ETM. Furthermore, the conductive oligomer is more conductive than the insulators as defined herein. Additionally, the conductive oligomers of the invention are to be distinguished from electroactive polymers, that themselves may donate or accept electrons.

In a preferred embodiment, the conductive oligomers have a conductivity, S, of from between about 10⁻⁶ to about 10⁴ Ω⁻¹ cm⁻¹, with from about 10⁻⁵ to about 10³ Ω⁻¹ being preferred, with these S values being calculated for molecules ranging from about 20 Å to about 200 Å. As described below, insulators have a conductivity S of about 10⁻⁷ Ω⁻¹ cm⁻¹ or lower, with less than about 10⁻⁸ Ω⁻cm⁻¹ being preferred. See generally Gardner et al., Sensors and Actuators A 51 (1995) 57-66, incorporated herein by reference.

Desired characteristics of a conductive oligomer include high conductivity, sufficient solubility in organic solvents and/or water for synthesis and use of the compositions of the invention, and preferably chemical resistance to reactions that occur i) during binding ligand synthesis (i.e. nucleic acid synthesis, such that nucleosides containing the conductive oligomers may be added to a nucleic acid synthesizer during the synthesis of the compositions of the invention, ii) during the attachment of the conductive oligomer to an electrode, or iii) during binding assays. In addition, conductive oligomers that will promote the formation of self-assembled monolayers are preferred.

The oligomers of the invention comprise at least two monomeric subunits, and can include homo- and hetero-oligomers, and include polymers. Generally, oligomers of the invention comprise charge neutral conjugated polymers, see generally U.S. Ser. No. 09/962,913, hereby incorporated by reference. Suitable conductive polymers include, but are not limited to, polypyrrole, polythiophene, polyaniline, polyfuran, polypyridine, polycarbazole, polyphenylene, poly(phenylenevinylene), polyfluorene, polyindole, derivatives thereof, co-polymers thereof, and combinations thereof. Preferably the conductive polymer is polypyrrole, polythiophene and polyaniline, and most preferable is polypyrrole. See generally U.S. Ser. No. 60/314,611, hereby incorporated by reference.

In “electron transfer mode”, the detection electrode comprises a self-assembled monolayer (SAM) comprising conductive oligomers. By “monolayer” or “self-assembled monolayer” or “SAM” herein is meant a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. A “mixed” monolayer comprises a heterogeneous monolayer, that is, where at least two different molecules make up the monolayer. The SAM may comprise conductive oligomers alone, or a mixture of conductive oligomers and insulators. As outlined herein, the efficiency of target analyte binding (for example, oligonucleotide hybridization) may increase when the analyte is at a distance from the electrode. Similarly, non-specific binding of biomolecules, including the target analytes, to an electrode is generally reduced when a monolayer is present. Thus, a monolayer facilitates the maintenance of the analyte away from the electrode surface. In addition, a monolayer serves to keep charged species away from the surface of the electrode. Thus, this layer helps to prevent electrical contact between the electrodes and the ETMs, or between the electrode and charged species within the solvent. Such contact can result in a direct “short circuit” or an indirect short circuit via charged species which may be present in the sample. Accordingly, the monolayer is preferably tightly packed in a uniform layer on the electrode surface, such that a minimum of “holes” exist. The monolayer thus serves as a physical barrier to block solvent accessibility to the electrode.

In a preferred embodiment, the detection electrode further comprises a capture binding ligand, preferably covalently attached. By “binding ligand” or “binding species” herein is meant a compound that is used to probe for the presence of the target analyte, that will bind to the target analyte. In general, for “electron transfer mode” embodiments described herein, there are at least two binding ligands used per target analyte molecule; a “capture” or “anchor” binding ligand (also referred to herein as a “capture probe”, particularly in reference to a nucleic acid binding ligand) that is attached to the detection electrode as described herein, and a soluble binding ligand, that binds independently to the target analyte, and either directly or indirectly comprises at least one ETM.

Generally, the capture binding ligand allows the attachment of a target analyte to the detection electrode, for the purposes of detection. As is more fully outlined below, attachment of the target analyte to the capture binding ligand may be direct (i.e. the target analyte binds to the capture binding ligand) or indirect (one or more capture extender ligands may be used).

In a preferred embodiment, the binding is specific, and the binding ligand is part of a binding pair. By “specifically bind” herein is meant that the ligand binds the analyte, with specificity sufficient to differentiate between the analyte and other components or contaminants of the test sample. However, as will be appreciated by those in the art, it will be possible to detect analytes using binding that is not highly specific; for example, the systems may use different binding ligands, for example an array of different ligands, and detection of any particular analyte is via its “signature” of binding to a panel of binding ligands, similar to the manner in which “electronic noses” work. The binding should be sufficient to allow the analyte to remain bound under the conditions of the assay, including wash steps to remove non-specific binding. In some embodiments, for example in the detection of certain biomolecules, the binding constants of the analyte to the binding ligand will be at least about 10⁻⁴ to 10⁻⁶ M⁻¹, with at least about 10⁻⁵ to 10⁻⁹ being preferred and at least about 10⁻⁷ to 10⁻⁹ M⁻¹ being particularly preferred.

As will be appreciated by those in the art, the composition of the binding ligand will depend on the composition of the target analyte. Binding ligands to a wide variety of analytes are known or can be readily found using known techniques. For example, when the analyte is a single-stranded nucleic acid, the binding ligand is generally a substantially complementary nucleic acid. Alternatively, as is generally described in U.S. Pat. Nos. 5,270,163, 5,475,096, 5,567,588, 5,595,877, 5,637,459, 5,683,867, 5,705,337, and related patents, hereby incorporated by reference, nucleic acid “aptomers” can be developed for binding to virtually any target analyte. Similarly the analyte may be a nucleic acid binding protein and the capture binding ligand is either a single-stranded or double-stranded nucleic acid; alternatively, the binding ligand may be a nucleic acid binding protein when the analyte is a single or double-stranded nucleic acid. When the analyte is a protein, the binding ligands include proteins (particularly including antibodies or fragments thereof (FAbs, etc.)), small molecules, or aptamers, described above. Preferred binding ligand proteins include peptides. For example, when the analyte is an enzyme, suitable binding ligands include substrates, inhibitors, and other proteins that bind the enzyme, i.e. components of a multi-enzyme (or protein) complex. As will be appreciated by those in the art, any two molecules that will associate, preferably specifically, may be used, either as the analyte or the binding ligand. Suitable analyte/binding ligand pairs include, but are not limited to, antibodies/antigens, receptors/ligand, proteins/nucleic acids; nucleic acids/nucleic acids, enzymes/substrates and/or inhibitors, carbohydrates (including glycoproteins and glycolipids)/lectins, carbohydrates and other binding partners, proteins/proteins; and protein/small molecules. These may be wild-type or derivative sequences. In a preferred embodiment, the binding ligands are portions (particularly the extracellular portions) of cell surface receptors that are known to multimerize, such as the growth hormone receptor, glucose transporters (particularly GLUT4 receptor), transferrin receptor, epidermal growth factor receptor, low density lipoprotein receptor, high density lipoprotein receptor, leptin receptor, interleukin receptors including IL-1, IL-2, IL-3, IL-4, IL-5, IL-6, IL-7, IL-8, IL-9, IL-11, IL-12, IL-13, IL-15 and IL-17 receptors, VEGF receptor, PDGF receptor, EPO receptor, TPO receptor, ciliary neurotrophic factor receptor, prolactin receptor, and T-cell receptors. Similarly, there is a wide body of literature relating to the development of binding partners based on combinatorial chemistry methods.

In this embodiment, when the binding ligand is a nucleic acid, preferred compositions and techniques are outlined in WO 98/20162; PCT/US98/12430; PCT/US98/12082; PCT/US99/01705; PCT/US99/01703; and U.S. Ser. Nos. 09/135,183; 60/105,875; and 09/295,691, all of which are hereby expressly incorporated by reference.

The method of attachment of the capture binding ligands to the attachment linker (either an insulator or conductive oligomer) will generally be done as is known in the art, and will depend on both the composition of the attachment linker and the capture binding ligand. In general, the capture binding ligands are attached to the attachment linker through the use of functional groups on each that can then be used for attachment. Preferred functional groups for attachment are amino groups, carboxy groups, oxo groups and thiol groups. These functional groups can then be attached, either directly or indirectly through the use of a linker, sometimes depicted herein as “Z”. Linkers are well known in the art; for example, homo-or hetero-bifunctional linkers as are well known (see 1994 Pierce Chemical Company catalog, technical section on cross-linkers, pages 155-200, incorporated herein by reference). Preferred Z linkers include, but are not limited to, alkyl groups (including substituted alkyl groups and alkyl groups containing heteroatom moieties), with short alkyl groups, esters, amide, amine, epoxy groups and ethylene glycol and derivatives being preferred, with propyl, acetylene, and C₂ alkene being especially preferred. Z may also be a sulfone group, forming sulfonamide linkages.

In this way, capture binding ligands comprising proteins, lectins, nucleic acids, small organic molecules, carbohydrates, etc. can be added.

A preferred embodiment utilizes proteinaceous capture binding ligands. As is known in the art, any number of techniques may be used to attach a proteinaceous capture binding ligand to an attachment linker. A wide variety of techniques are known to add moieties to proteins.

A preferred embodiment utilizes nucleic acids as the capture binding ligand. While most of the following discussion focuses on nucleic acids, as will be appreciated by those in the art, many of the techniques outlined below apply in a similar manner to non-nucleic acid systems as well.

Thus, one end of the attachment linker is attached to a nucleic acid (or other binding ligand), and the other end (although as will be appreciated by those in the art, it need not be the exact terminus for either) is attached to the electrode.

In a preferred embodiment, for “electron transfer mode” systems that utilize “sandwich” type assays, the compositions further comprise a solution or soluble binding ligand. Solution binding ligands are similar to capture binding ligands, in that they bind, preferably specifically, to target analytes. The solution binding ligand may be the same or different from the capture binding ligand. Generally, the solution binding ligands are not directed attached to the surface. The solution binding ligand either directly comprises a recruitment linker that comprises at least one ETM, or the recruitment linker binds, either directly or indirectly to the solution binding ligand.

Thus, “solution binding ligands” or “soluble binding ligands” or “signal carriers” or “label probes” or “label binding ligands” with recruitment linkers comprising covalently attached ETMs are provided. That is, one portion of the label probe or solution binding ligand directly or indirectly binds to the target analyte, and one portion comprises a recruitment linker comprising covalently attached ETMs. The terms “electron donor moiety”, “electron acceptor moiety”, and “ETMs” (ETMs) or grammatical equivalents herein refers to molecules capable of electron transfer under certain conditions. It is to be understood that electron donor and acceptor capabilities are relative; that is, a molecule which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds in the present invention. Preferred ETMs include, but are not limited to, transition metal complexes, organic ETMs, and electrodes.

In a preferred embodiment, the ETMs are transition metal complexes. Transition metals are those whose atoms have a partial or complete d shell of electrons. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinum (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinum, cobalt and iron.

L are the co-ligands, that provide the coordination atoms for the binding of the metal ion. As will be appreciated by those in the art, the number and nature of the co-ligands will depend on the coordination number of the metal ion. Mono-, di- or polydentate co-ligands may be used at any position. Thus, for example, when the metal has a coordination number of six, the L from the terminus of the conductive oligomer, the L contributed from the nucleic acid, and r, add up to six. Thus, when the metal has a coordination number of six, r may range from zero (when all coordination atoms are provided by the other two ligands) to four, when all the co-ligands are monodentate. Thus generally, r will be from 0 to 8, depending on the coordination number of the metal ion and the choice of the other ligands.

In one embodiment, the metal ion has a coordination number of six and both the ligand attached to the conductive oligomer and the ligand attached to the nucleic acid are at least bidentate; that is, r is preferably zero, one (i.e. the remaining co-ligand is bidentate) or two (two monodentate co-ligands are used).

As will be appreciated in the art, the co-ligands can be the same or different. Suitable ligands fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lₘ). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam), EDTA, EGTA and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp73-98), 21.1 (pp. 813-898) and 21.3 (pp 915-957), all of which are hereby expressly incorporated by reference.

Suitable sigma donating ligands using carbon, oxygen, sulfur and phosphorus are known in the art. For example, suitable sigma carbon donors are found in Cotton and Wilkenson, Advanced Organic Chemistry, 5 th Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson.

The oxygen, sulfur, phosphorus and nitrogen-donating ligands are attached in such a manner as to allow the heteroatoms to serve as coordination atoms.

In a preferred embodiment, organometallic ligands are used. In addition to purely organic compounds for use as redox moieties, and various transition metal coordination complexes with δ-bonded organic ligand with donor atoms as heterocyclic or exocyclic substituents, there is available a wide variety of transition metal organometallic compounds with π-bonded organic ligands (see Advanced Inorganic Chemistry, 5th Ed., Cotton & Wilkinson, John Wiley & Sons, 1988, chapter 26; Organometallics, A Concise Introduction, Elschenbroich et al., 2nd Ed., 1992, VCH; and Comprehensive Organometallic Chemistry II, A Review of the Literature 1982-1994, Abel et al. Ed., Vol. 7, chapters 7, 8, 10 & 11, Pergamon Press, hereby expressly incorporated by reference). Such organometallic ligands include cyclic aromatic compounds such as the cyclopentadienide ion [C₅H₅(−1)] and various ring substituted and ring fused derivatives, such as the indenylide (−1) ion, that yield a class of bis(cyclopentadieyl) metal compounds, (i.e. the metallocenes); see for example Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to either the ribose ring or the nucleoside base of nucleic acid. Other potentially suitable organometallic ligands include cyclic arenes such as benzene, to yield bis(arene)metal compounds and their ring substituted and ring fused derivatives, of which bis(benzene)chromium is a prototypical example, Other acyclic π-bonded ligands such as the allyl(-1) ion, or butadiene yield potentially suitable organometallic compounds, and all such ligands, in conjunction with other π-bonded and δ-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene are derivatized.

As described herein, any combination of ligands may be used. Preferred combinations include: a) all ligands are nitrogen donating ligands; b) all ligands are organometallic ligands; and c) the ligand at the terminus of the conductive oligomer is a metallocene ligand and the ligand provided by the nucleic acid is a nitrogen donating ligand, with the other ligands, if needed, are either nitrogen donating ligands or metallocene ligands, or a mixture.

In addition to transition metal complexes, other organic electron donors and acceptors may be covalently attached to the nucleic acid for use in the invention. These organic molecules include, but are not limited to, riboflavin, xanthene dyes, azine dyes, acridine orange, N,N′-dimethyl-2,7-diazapyrenium dichloride (DAP2+), methylviologen, ethidium bromide, quinones such as N,N′-dimethylanthra(2,1,9-def:6,5,10-d′e′f′)diisoquinoline dichloride (ADIQ2+); porphyrins ([meso-tetrakis(N-methyl-x-pyridinium)porphyrin tetrachloride], varlamine blue B hydrochloride, Bindschedler's green; 2,6-dichloroindophenol, 2,6-dibromophenolindophenol; Brilliant crest blue (3-amino-9-dimethyl-amino-10-methylphenoxyazine chloride), methylene blue; Nile blue A (aminoaphthodiethylaminophenoxazine sulfate), indigo-5,5′,7,7′-tetrasulfonic acid, indigo-5,5′,7-trisulfonic acid; phenosafranine, indigo-5-monosulfonic acid; safranine T; bis(dimethylglyoximato)-iron(II) chloride; induline scarlet, neutral red, anthracene, coronene, pyrene, 9-phenylanthracene, rubrene, binaphthyl, DPA, phenothiazene, fluoranthene, phenanthrene, chrysene, 1,8-diphenyl-1,3,5,7-octatetracene, naphthalene, acenaphthalene, perylene, TMPD and analogs and subsitituted derivatives of these compounds.

In one embodiment, the electron donors and acceptors are redox proteins as are known in the art. However, redox proteins in many embodiments are not preferred.

In a preferred embodiment, the detection module comprises an optical detection such as laserinduced fluorescence or UV-absorbance. An example of basic confocal epifluorescence set up for high sensitivity is found in Jiang et al., Biosens. Bioelectron. 2000, 14, 861-869. An improved UV-detection method for microfluidic device can be found in Salimi-Moosavi et al., Electrophoresis 2000, 21, 1291-1299.

In a preferred embodiment, the detection module comprises a mass spectrometry apparatus such as Matrix-Assisted Laser Desorption/Ionization (MALDI) and electrospray ionization-mass spectrometry (ESI-MS). The term “MALDI” is used herein to refer to a process wherein analyte is embedded in a solid or crystalline “matrix” of light-absorbing molecules e.g., nicotinic, sinapinic, or 3-hydroxypicolinic acid), then desorbed by laser irradiation and ionization from the solid phase into the gaseous or vapor phase, and accelerated as intact molecular ions towards a detector. The integration of MALDI into a microfluidic device is taught by U.S. Pat. No. 5,716,826.

In a preferred embodiment, the detection is carried out while the target analytes are captured within the microchannel. For example, the target analyte can be labeled by both magnetic labels and a detection label such as a fluorescent group. When the target analyte is captured in the magnetic microchannel, preferably on the lateral surfaces of the channels, the detection labels attached to the target analytes can be detected by a detection device such as a fluorescent microscope.

In a preferred embodiment, optics are included near the channel, so that light can be coupled into and out of the channel. For example, diffractive optical lenses, beam splitters, and other optical elements can be fabricated into the channel. See Quake et al., Science 290, 1536.

The devices of the invention are generally made as outlined herein and using techniques well known in the art.

In a preferred embodiment, a device comprising “embedded channels” is made by modification of conventional techniques for fabricating microchannel structures, for example, the technique disclosed in U.S. Pat. No. 6,176,962. Suitable substrates for this embodiment include, but is not limited to, plastics, PDMS and other materials, as outlined above. In a preferred embodiment, with devices prepared from a plastic material, a silica mold master which is a negative for the channel structure can be prepared by etching or laser micromachining. A polymer precursor is first impregnated with magnetic beads. The beads are generally deposited in a monolayer or near a monolayer at the channel surface. Their higher density generally keeps them in place and at the channel surface if the beads are on top of the channel mold. The impregnated precursor can then be thermally cured or photopolymerized between the silica master and support planar plate, such as a glass plate. After the planar substrate has been fabricated, a cover plate may be placed over, and sealed to, the surface of the substrate, thereby forming an integrated device. The cover plate may be sealed to the substrate using any convenient means, including ultrasonic, welding, adhesive, etc. Alternatively, the planar substrate can be sealed with a flexible cover as described in PCT US01/02664, incorporated herein by reference. It should be clear to the skilled in art that the cover plate may also be prepared from a precursor impregnated with magnetic beads, thus making channels surrounded by the magnetic beads.

In a preferred embodiment, a device comprising “coated channels” is made by modification of conventional techniques for fabricating microchannel structures. For example, a substrate with microchannel can be fabricated using any convenient means, such as molding and casting techniques. The microchannel are then coated with magnetic beads impregnated into a coating material. The coating material includes, but is not limited to, polycarbonate, polypropylene, acrylics, epoxies, PDMS, etc, even agarose or acrylamide. Upon coating, the substrate is then sealed with a cover plate, as described above. One advantage of this technique includes that fabrication can be done for any pre-existing channel, for example, injection-molded devices.

In a preferred embodiment, the device comprising magnetic microchannel filled with magnetic beads are made using techniques well known in the art. For example, with devices prepared from a plastic material, a silica mold having at least one raised ridges for the position of the magnetic microchannel can be prepared. Next, a polymer precursor formulation can be thermally cured or photopolymerized between the silica master and support planar plate. After the planar substrate is fabricated, filled-channels, prepared in a separate device, can then be placed into the cavity and connected to other parts of the device. Finally, a cover plate is placed over the planar substrate and sealed to the substrate as outlined above, thereby forming an integrated device. Alternatively, the channels are first made with conventional techniques, and magnetic beads are subsequently filled into the magnetic channel.

In a preferred embodiment, the magnetic microchannel comprises gradient inducing features coated with magnetic materials. The magnetic material is preferably electroplated onto gradient-inducing features, however, other methods such as sputtering and evaporation may be used. Similar fabrication methods to those used to fabricate channels, discussed above, may be used to fabricate gradient inducing features including photolithography techniques, wet and dry etching, laser drilling, etc. Gradient inducing features may be fabricated directly. Alternatively, a ‘negative mold’ may be fabricated and used to form the gradient inducing features, for example using injection molding techniques.

In a preferred embodiment, shown in FIG. 7, negative mold 71 comprising ridges 72 and 74 (defining valley 73) and pits 76 and 78 is fabricated from silicon using an etchant comprising hydrofluoric acid (HF), nitric acid (HNO₃), and acetic acid (CH₃COOH) in a ratio of 1:3:8, generally known as HNA. A layer of SiO₂ is preferably used to mask the silicon, although other materials, such as silicon nitride may be used. The masking material is removed above valley 73, pit 78 and pit 76. Briefly, exposure to HNA results in isotropic etching, that is etching that proceeds both down into the silicon and laterally under the masking material. The etching rate is affected by the size of the mask opening. The distance between ridges is significantly greater than the width of pits, accordingly the area between ridges, valley 73, is etched deeper than the pits 76 and 78. A preferred embodiment is described in greater detail in the example below. It is to be understood that these measurements are by way of example, and that the inventive process would apply to a variety of ridge and pit dimensions. After negative mold 70 is formed, it may be used in, for example, a injection molding process to generate a device comprising a microchannel containing a dome structure. Ridges 72 and 74 correspond to resultant microchannels, and pits 78 and 76 to a dome within each microchannel.

Once made, the devices of the invention find use in a number of applications.

In principle, any biological samples that contain magnetic components or components that can be magnetically labeled can be processed by the microchannel. The inventive method for the use of the present device generally comprises (1) provide a biological sample containing a component labeled with a magnetic label; (2) introducing the biological sample to a microfluidic device comprising a magnetic microchannel under a condition whereby the labeled components are retained in the magnetic microchannel, while those that not labeled flow through. If the target analytes are retained in the channel, they can be washed at least once while retained in the channel. After the optional washing step, the target analytes can either be directly detected in the channel, or eluted from the microchannel for further processing and/or detection.

Target analytes, or other magnetic or magnetically-labeled particles, are retained in the channel as they are drawn to an area of high magnetic field strength within the channel. In a preferred embodiment, an area of high magnetic field strength is provided by magnetic beads within a wall of the channel. In yet another embodiment, an area of high magnetic field strength is provided by gradient inducing features within the channel, as described above. Thus, magnetic or magnetically-labeled particles may be retained in a channel despite surrounding fluid flow, as the magnetic or magnetically-labeled particles are attracted to areas of high magnetic field strength. Similarly, magnetic or magnetically-labeled particles may be separated within the channel according to their magnetic response.

In a preferred embodiment, the components in the sample are labeled in a labeling chamber integral to the inventive device, as outlined above. In another embodiment, they can be labeled in a separate device prior to the processing by the present device. Alternatively, the biological sample contains components that are intrinsically magnetic, i.e., possessing magnetic property without being attached to a magnetic label.

In a preferred embodiment, the biological sample is introduced into the magnetic microchannel through the sample inlet port. The amount of sample to be introduced each time depends on the concentration of the magnetic or magnetically labeled component in the biological sample. To achieve a maximum capturing efficiency, it is preferred that the total amount of the labeled components that is introduced into the magnetic microchannel does not exceed the amount that will saturate all sections of the channel comprising magnetic beads or gradient inducing features.

The sample can be introduced into the channel as a continuous flow though the channel. The flow rate of the sample can be slow, for example less than 1 mm/sec average velocity, for a greater capturing efficiency. Alternatively, the sample outlet port and the disposal port can be closed temporarily during the loading of the sample. Upon loading of the sample, the flow can also stopped temporarily to allow the magnetic or magnetically labeled component to be captured. After the capturing step, the uncaptured components are then disposed or collected as desired.

In a preferred embodiment, usually when the target analytes are retained in the magnetic microchannel, the channels are washed at least once, by running a sufficient volume of washing buffers through the magnetic microchannel. Various buffer can be used as a washing buffer, as long as they don't disrupt the binding between the target analytes and the binding ligand on the magnetic particle. For instance, phosphate buffered saline (PBS) can be conveniently used. The buffer can either be introduced into the microchannel through the sample inlet port, or through a separate fluidic inlet port. The resultant wash solution can then be disposed through the sample outlet port, or more preferably, the disposal port.

The washing buffer can be introduced into the channel in separate batches, each batch having a volume of or more than the chamber volume. Alternatively, the buffer can run through the channel as a continuous flow. When a continuous flow of washing buffer is running through the magnetic channel, one wash is achieved by running one chamber volume of the washing buffer through the channel. Similarly, more washes is achieved by running more than one chamber volume of the washing buffer through the channel.

In a preferred embodiment, the target analytes are eluted from the magnetic microchannel. The elution can be achieved in a variety of ways. For example, the target analytes can be eluted along with the magnetic labels by introducing magnetic ferrofluids into the channel or by reversing the polarity of the electromagnets that provide the magnetic field in the channel. Alternatively, the target analytes can be eluted by a releasing reaction, as outlined above.

In a preferred embodiment, the elution of the target analyte is achieved by supplying the magnetic microchannel with magnetic ferrofluid, i.e., a fluid containing a suspension or dispersion of particles with higher magnetization than those which are retained. The ferrofluid will effectively displace the retained materials in the magnetic microchannel or alter the characteristics of the overall magnetic environment in the magnetic microchannel, and thus result in the flow of the retained particles through the sample outlet port.

In a preferred embodiment when the magnetic microchannel comprise external electromagnets (in the cased of embedded channel, coated channel, filled channel, and channel comprising a gradient inducing feature), elution of the target analyte can be achieved by reversing the polarity of the electromagnets. The change of the magnetic environment will then result in the release of the labeled material from the channel.

In a preferred embodiment, elution of the target analyte is achieved by releasing the target analytes from the magnetic labels, under a condition that disrupts the binding between the target analyte and the binding ligand on the magnetic particle, as fully described above.

In a preferred embodiment, the target analytes retained in the magnetic channel are further subjected to chemical reactions inside the channel, as has been described above for the reaction module. The products of such reactions can then be released from the channel and detected. If the reaction products are still attached to the magnetic labels, and are thus retained in the magnetic microchannel, they can either be eluted from the channel by the methods described above, or detected directly within the magnetic microchannel.

In a preferred embodiment, the target analytes or reaction products resulting from the target analytes are directly detected while they are retained in the magnetic microchannel. Preferably, the target analyte or the reaction product to be detected contain detection labels. The detection labels include, but is not limited to, fluorescent, chemiluminescent and radioactive compounds, compounds which have distinct or recognizable light scattering or other optical properties, and compounds which are only detectable upon binding to the characteristic determinant. It should be clear to those skilled in the art that when the target analyte is simultaneously labeled with a magnetic label and a detection label, it is necessary that the binding ligand on the magnetic label recognize a separate epitope on the target analyte from the one recognized by the detection label.

In a preferred embodiment, more than one target analyte can be sorted by a single processing in the magnetic microchannel. For example, the mixture is treated with magnetic particles conjugated to anti-A which have high magnetic susceptibility and particles conjugated to anti-B which have low magnetic susceptibility. The labeled mixture is then applied to the device and a magnetic field strength sufficient to retain both A and B associated magnetic particles is supplied. In elution, the magnetic field strength or the magnetization of the eluting ferrofluid are altered so as to release particles which are associated with B but not those associated with A, thus effecting a separation of A and B.

In principle, any number of components in a sample can be labeled with magnetic particles of differing magnetizations by treating various groups of labels with a different specific binding ligand complementary to a chosen component of the mixture. As described above, the labeling can be done in a single labeling reaction, or, more preferably, in separate reactions. Each component will then uniquely react with one representative composition of a particular magnetization. The labeled mixture, when subjected to the magnetic microchannel results in a chromatographic pattern of components separated according to the magnetization of the particles with which they are conjugated. Once processed in the microchannel, the target analytes can then be further processed and/or detected, either together or separately.

In a preferred embodiment, particles of differing magnetizations are separated by providing a plurality of gradient inducing features. By varying the dimension of each gradient inducing feature, several regions of differing magnetic field strengths are established within the magnetic channel. Magnetic or magnetically-labeled particles are sorted into these areas of differing magnetic field strength according to their particular magnetic response.

The present invention is applicable for a variety of purposes. For example, the device can be used to isolate and/or detect cells, nucleic acids, or proteins. The target analytes can be enriched and/or purified by being captured to the magnetic microchannel and thus separated from the rest of the sample. Alternatively, the target analytes can be separated from other components that are retained in the channel. Advantageously, the magnetic microchannel in the present invention can easily be washed after each use, so that a single microfluidic device can be reused, either to detect the same kind of target analytes, or a different kind of target analytes.

In a preferred embodiment, the microfluidic devices of the invention are used to isolate and/or detect a particular kind of cells. Suitable cells are described above. In some embodiments, the presence of a certain kind of cells can be determined for diagnosis or other analytical purposes. In some other embodiments, cells can be isolated so that the target analytes within the cells can be further processed and detected.

Depending on the particular configuration of the device, target cells are first separated from other components in the cell separation module before they are labeled in a labeling chamber and processed in the magnetic microchannel. However it is also possible to first label the cells in the sample, separate out cells from other components in a cell separation module, and then process the cell mixture in the magnetic microchannel. A cell separation step prior to a labeling reaction allows the enrichment of the target cells in the sample, and thus facilitate the labeling reaction. Similarly, a cell separation step prior to the processing in the magnetic microchannel increases the capturing efficiency of the target cells. On the other hand, the magnetic microchannel itself may serve the purpose of a cell separation module for subsequent processes.

The labeling of the cells with magnetic labels are outlined above. The magnetic labels contain binding ligands that recognize a specific epitope on the cell surface. The labeled cells can then be captured in the magnetic channel and be separated from the rest of the sample.

In a preferred embodiment, the target cells are simultaneously labeled with a magnetic label and a detection label, so that they can be directly detected while captured in the magnetic microchannel. The addition of a detection label on the cell can also be carried out within the channel, while the cells are capture, for example using a method similar to the immunostaining technique. Alternatively, the target cells may be detected directly without a detection label. For instance, the target cells may express a GFP and thus can be detected by a fluorescence microscope.

In a preferred embodiment, the target cell are subjected to a cell lysis reaction while captured in the channel. In this embodiment, lysis buffer are introduced from a buffer inlet port under a condition that a substantial amount of cells can be lysed. The resultant cell lysates can then be collected from a sample outlet port. The cell lysated can be subjected to another round of magnetic labeling and processing in a magnetic microchannel. Alternatively, the lysate can be processed in other modules of the device.

In a preferred embodiment, cells captured in the magnetic channel are eluted from the channel. When intact cells are to be detected, the cells are eluted by magnetic ferrofluid, reversal of the electromagnets, or a releasing reaction that does not disrupt the integrity of the cell. The eluted cells, further released from the magnetic label if necessary, are then detected. The detection can be achieved by routine methods such as fluorescent microscope, cell counting and sorting devices, etc.

In a preferred embodiment, the microfluidic devices of the invention are used to detect target nucleic acids. In this embodiment, target nucleic acids are labeled by magnetic labels containing a binding ligand such as a complementary nucleic acid, a nucleic acid binding protein, etc. The labeled nucleic acids are then captured by the microchannel and separated from the rest of the sample.

Optionally, the target nucleotide in the sample can be amplified by means of in vitro amplification reactions, such as the PCR techniques and other techniques fully disclosed above. Amplifying the target nucleic acids prior to the processing in the magnetic microchannel allows a more efficient capturing by the channel. On the other hand, enriching the nucleic acids by the magnetic microchannel prior to an amplification reaction provides more rapid and more accurate templatedirected synthesis by the polymerase. The use of such in vitro amplification methods is optional in the methods of the invention, which makes the present invention advantageous. A target nucleic acid sequence that is rare in the sample normally requires an amplification step to generate sufficient signal to be detected. The amplification methods, such as PCR, typically produces errors in the target nucleic acid sequence, thus raises problems when the sequences of the target nucleic acids are to be accurately determined. The magnetic microchannel processing allows a fast and specific enrichment of the target nucleic acids, thus allows a detection of the target sequence without amplification.

When target nucleic acids are amplified prior to the processing in the magnetic microchannel, the primers that are used in the amplification reaction can be labeled with magnetic labels, so that the amplification products are automatically labeled in the amplification reaction. Alternatively, the amplification products are subsequently labeled in a separate labeling reaction. The labeled nucleic acids are then introduced to and captured in the magnetic microchannel.

In a preferred embodiment, the nucleic acid that are captured in the channel are amplified inside the magnetic channel. The amplified products are then allowed to flow out of the magnetic microchannel. In this embodiment, a thermal unit is placed properly next to the magnetic microchannel, so that the channel chamber can serve as a reaction chamber for amplification reactions. Alternatively, the eluted target nucleic acids, released from the magnetic label if necessary, can also be subsequently amplified in a reaction module.

In a preferred embodiment, the target nucleic acids are detected while they are captured in the microchannel. For example, the targets may be simultaneously labeled by a detection label and a magnetic label, as described above for “sandwich” type assays. Similarly, the target nucleic acids can labeled inside the magnetic channel. Labeled target nucleic acid can then be detected by a detection device.

In a preferred embodiment, the microfluidic devices of the invention are used to detect a target protein. In this embodiment, target proteins are labeled by magnetic labels that contain specific binding ligands such as antibodies specific for the target protein, or other proteins, peptides, or small molecules that can be specifically recognized by the target proteins, as fully outlined above. The labeled proteins can then be captured by the magnetic microchannel and separated from the rest of the sample.

As outlined above for the detection of cells, proteins can be simultaneously labeled by another detection label so that they can be detected while captured in the channel or immediately after they are eluted from the channel. For example, two monoclonal antibodies recognizing two different epitopes on the protein can be used for the two kinds of labels. The addition of a detection label can either be done in a reaction module or a labeling chamber prior to the processing in the magnetic microchannel, or inside the channel. In some cases, when the protein can be detected without a detection label, for instance when the protein is a GFP protein or a GFP fusion protein, the addition of a detection label would not be necessary.

In a preferred embodiment, antibodies or fragments of antibodies are used as detection label. The labeling can be done under conditions well known in the art. (similar to immunoassay)

In a preferred embodiment when the protein is an enzyme, an enzymatic reaction can be carried out inside the magnetic microchannel by introducing its substrate into the channel. The reaction products can then be detected within the channel. Alternatively, the reaction product can be allowed to flow through the magnetic microchannel and detected afterwards, for instance by a spectrometer directly linked to the sample outlet port.

In a preferred embodiment, the proteins that are processed in the magnetic microchannel are further subjected to separation. For example, the target protein may be labeled by some ubiquitous label and processed in the magnetic microchannel. The processed protein sample is then subjected to another round of magnetic labeling and processing. Alternatively, the processed protein sample are further processed by a separation module, such as electrophoresis.

In a preferred embodiment, the eluted target proteins are modified or cleaved in a reaction module before they are detected. For example, target proteins may be cleaved into peptide fragments by CNBr, or hydrolyzed by enzymes. The peptide fragments are then subjected to mass-spectrometry analysis, as described above. The modification or cleavage reactions can also be carried out in the magnetic microchannel, while the proteins are captured in the channel.

In a preferred embodiment, the biological sample is “cleaned up” by going through the magnetic microchannel, i.e., some undesired components, rather than target analytes, are labeled by magnetic labels and retained by the magnetic channel. Generally but not necessarily, a relatively nonspecific binding ligand is used to capture the undesired components. For example, when some kind of bacteria need to be removed from a biological sample, a mixture of antibodies for the bacteria to be removed can be used as binding ligands.

When the samples are introduced into the magnetic channel, the labeled components will be retained in the channel, while the rest of the sample, including the target analytes will pass through. The then “cleaned-up” sample can be subjected to subsequent processing, which may include another round of magnetic labeling and processing of either the target analyte or other components. Alternatively, the target analytes are further processed in the separation module and reaction modules before they are detected.

The above-described “clean up” steps can be repeated several rounds in a single processing event. This can be done by subjecting the flow-though portion of the sample to the same magnetic microchannel. Alternatively, the sample can be “cleaned up” by going through several consecutively linked magnetic microchannel.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

### Fabrication Example for 50 μm High Ridge Structures Compression Molded Into Polycarbonate Structures Using an Etched Silicon Stamper

Silicon Stamper Fabrication

A plastic replication technique was implemented to construct ridge microstructures inside plastic microchannels. The polycarbonate microchannels were fabricated by compression molding using Carver hydraulic laboratory presses (Carver, Inc., Wabash, Ind.). Silicon (Si) stamper was used as a mold to transfer the channel/ridge patterns into the plastic. The Si stamper was fabricated using standard photolithographic procedures followed by a KOH anisotropic etching process. FIG. 8 shows a schematic of the anisotropic etched Si structure. Note that the pyramidal grooves are transformed into ridge microstructures in the plastic chip after the plastic compression molding. Si is a crystal substrate that has different crystal planes. KOH (alkali hydroxide) is an anisotropic etchant that etches much faster at (100) and (110) planes than at (111) plane, resulting in pyramidal grooves, such as groove 900, with 54.74° (111) sidewall angles (angle 910) relative to the surface in the Si substrate.

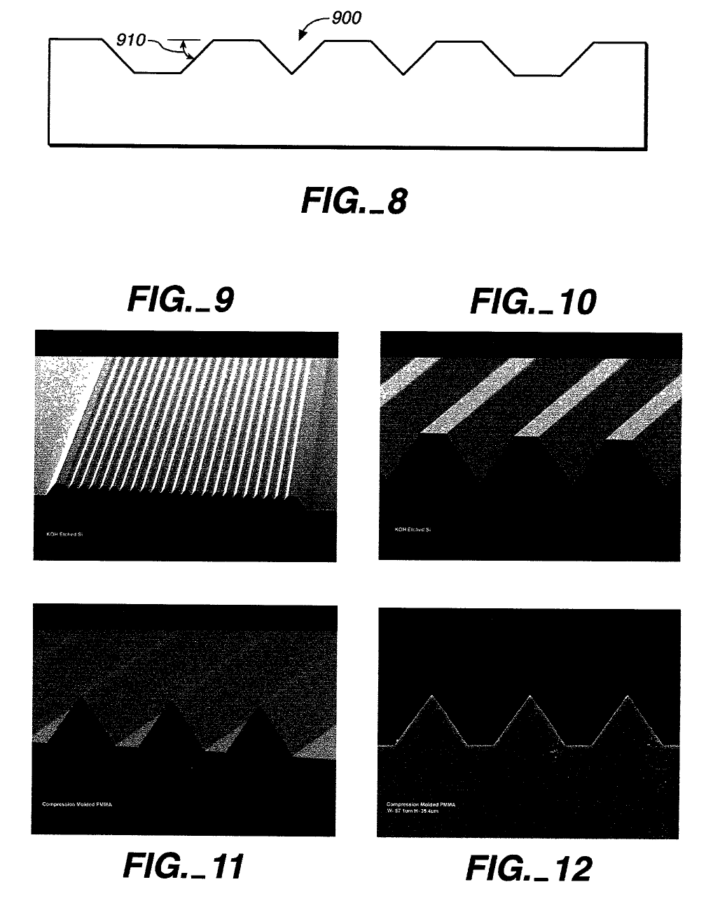

During the KOH etching, a 1 μm thick protective coating (mask for KOH etching) of Si₃N₄ was first deposited on a silicon (100) wafer using low-pressure chemical vapor deposition (LPCVD). A 500 Å film of chromium was then deposited using a sputtering system at 300 watts and a pressure of 10 mtorr using argon at a flow rate of 50 sccm for 3 minutes. On the top side of the wafer, the chromium was patterned using a chromium etchant (CEN-300, Microchrome Technology Inc, San Jose, Calif.) for 1.5 minutes, and the Si₃N₄was etched by reactive ion etching (RIE) at 150 watts and a pressure of 50 mtorr using CF₄ at a flow rate of 50 sccm for 15 minutes. The Si wafer was then etched in a bath with 22.5% concentration of KOH at 75° C. for 35 min. The resulting channel is 1 mm wide and 50 μm deep. The pyramidal grooves are 50 μm wide and 50 μm deep (see FIGS. 9 and 10). FIGS. 9 and 10 are scanning electron microscope (SEM) images of the anisotropic etched Si structure used to mold the plastic substrate.

Compression Molding

Following the etching of the Si stamper, the stamper was used as a mold to fabricate plastic microchannels with ridge microstructures. During the compression molding, a 5-mm-thick glass wafer was placed on the lower platen to provide a flat, smooth foundation surface. A 5-cm separation was established between the upper and lower platens. The silicon stamper was then placed on the glass wafer. The system was heated to 188° C. A predetermined amount of polycarbonate pellets (Aldrich) was placed in the center of the silicon stamper, and a blank nickel wafer was then placed on top of the polycarbonate pellets. The upper platen was lowered into contact with the blank nickel wafer and was then gradually compressed against the polycarbonate pellets as they melted. When the formed polycarbonate layer reached 1 mm in thickness, the two hot plates were separated, and the polycarbonate wafer and silicon stamper assembly were removed from the hydraulic press to air cool for ninety seconds. After cooling, the molded chip was demolded from the silicon stamper and the blank nickel plate. The entire molding process took approximately three minutes. The plastic microchannel with ridge microstructures is shown in FIGS. 11 and 12, which represent SEM images of the compression-molded plastic microchannel with ridge microstructures.

Electroplating

The molded structure was first sputtered with a metal seed layer of 100 angstroms Titanium-tungsten followed by 1000 angstroms of gold. The initial 100 angstroms of Ti—W is critical for adhesion to polymer substrates. A mask was used such that only the areas to be electroplated were sputtered. Following deposition of the seed layer, 80% nickel 20% iron alloy electroplating was performed with the following parameters: 1) Electroplating solution composition-200 g/L nickel chloride, 4 g/L ferrous chloride, 25 g/L boric acid, 1 g/L saccharin, 0.4 g/L sodium lauryl sulfate; 2) Operating conditions-pH 3, temperature 30 C, current density 2 A dm⁻²

Time of deposition will depend on desired layer thickness. For a 50 μm thick layer, plating duration was about 2 hours.

For the structure detailed above, calculations show gradient strength in the vertical direction at the tips of the ridges was on the order of >10,000 T/m in an external magnetizing field of 0.3 T. This gradient falls off however to near 0 just 50 μm from the tips.

For deeper channels it will be desirable in high flow applications to fabricate larger saw-toothed features beneath and perpendicular to the original smaller ridges, typically about 0.5 to 1 mm apart. Grooves are cut in the substrate using a CO₂ engraving or excimer laser. Substrate material is ablated away until the original plating is exposed. Typical grooves are 300 μm wide at the base and 50 μm wide at the tip. The new grooves are then plated as before but with a longer plating time (>8 hrs) such that the grooves fill in and become solid. In this way it is possible to maintain useful separation gradients on the order of ˜500 T/m at the far end of a 250 μm deep channel for the example given.

Once plating is complete the channel can be integrated with other components or used separately for direct detection. Depending on application, the channel is bonded to a top section comprising the substrate or microscope cover glass (for applications requiring viewing of the captured elements) or another magnetic channel.

### Example 2

### Fabrication of Microchannels With Dome Microstructures

A plastic replication technique was also implemented to construct micro-dome structures inside plastic microchannels. The polycarbonate microchannels were fabricated by compression molding using Carver hydraulic laboratory presses (Carver, Inc., Wabash, Ind.). A Silicon (Si) stamper was used to transfer the channel patterns into the plastic. The Si stamper was fabricated using standard photolithographic procedures followed by an isotropic wet etching process. A mixture of hydrofluoric acid (HF), nitric acid (HNO₃), and acetic acid (CH₃COOH) in a ratio of 1:3:8, also referred as “HNA”, is used as the etchant. The HNO₃ drives the oxidation of the silicon, while fluoride ions from HF then form the soluble silicon compound H₂SiF₆. The acetic acid, which is much less polar than water (smaller dielectric constant in the liquid state), helps prevent the dissociation of HNO₃ into NO₃⁻ or NO₂⁻, thereby allowing the formation of the species directly responsible for the oxidation of silicon. The overall reaction is as follows:

18HF+4 HNO₃+3Si→2 H₂SiF₆+4NO+8 H₂O

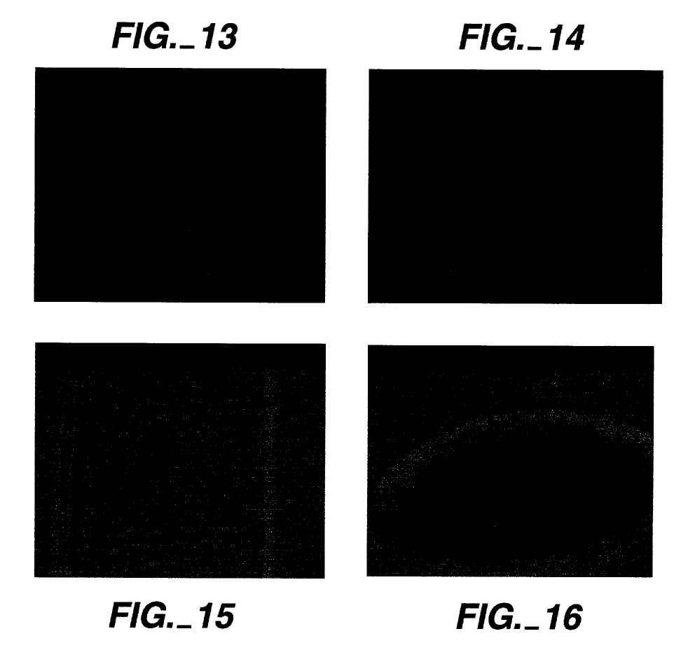

We used a thin layer of SiO₂ as a mask to etch Si. The etch rate of the Si using HNA etchant is ˜1 μm/min. One parameter to note of this isotropic etching process associated with the Si etching rate is the dissolution of the reaction products into the solution. If the reaction products can be transported quickly into the solution and the fresh etchant solution can be replenished and moved into the etching area rapidly, the Si etching rate is high. Otherwise, the etching rate can be very slow. We utilize this mechanism to achieve different etch rates at different locations. The areas between channels (which are ridge structures as shown in FIG. 7, are larger than the areas of dome arrays (which are pit structures here). The solution can easily move in and out of the channel areas as compared to the smaller pit areas. As a result, the Si in the areas between channels is etched twice as fast as Si in the pit areas. The resulting ridge (channel) is 40 μm high, while the pits are 20 μm deep (see FIGS. 13 and 14). FIGS. 13 and 14 show SEM images of pit structures of the isotropic etched Si stamper.

During the compression molding, a 5-mm-thick glass wafer was placed on the lower platen to provide a flat, smooth foundation surface. A 5-cm separation was established between the upper and lower platens. The silicon stamper was then placed on the glass wafer. The system was heated to 188° C. A predetermined amount of polycarbonate pellets (Aldrich) was placed in the center of the silicon stamper, and a blank nickel wafer was then placed on top of the,polycarbonate pellets. The upper platen was lowered into contact with the blank nickel wafer and was then gradually compressed against the polycarbonate pellets as they melted. When the formed polycarbonate layer reached 1 mm in thickness, the two hot plates were separated, and the polycarbonate wafer and silicon stamper assembly were removed from the hydraulic press to air cool for ninety seconds. After cooling, the molded chip was demolded from the silicon stamper and the blank nickel plate. The entire molding process took approximately three minutes. SEM images of a channel structure with micro-dome arrays obtained in compression molding process is shown in FIGS. 15 and 16. The channel is 40 μm deep, while the domes are 20 μm high.

Nickel-iron plating was accomplished as for the previous example. The resulting field gradients in a 0.3 T vertical field for the given example with a 100 μm thick plating can be expected to be around>1000 T/m near the tops of the domes.

We claim:

1. A microfluidic device comprising a solid support comprising:
a) a sample inlet port;
b) at least one microchannel comprising at least one section with walls comprising magnetic beads and an inner diameter devoid of said beads;
c) a sample outlet port.

2. A device according to claim 1 wherein said magnetic beads are embedded in said walls.

3. A device according to claim 1 wherein said magnetic beads are coated onto the inner surface of said walls.

4. A device according to claim 1 wherein said magnetic beads are of a uniform size.

5. A device according to claim 1 wherein said magnetic beads are of non-uniform size.

6. A device according to claims 1-5 wherein said magnetic beads are ferromagnetic.

7. A device according to claims 1-5 wherein said magnetic beads are permanently magnetized.

8. A device according to claims 1-5 wherein said magnetic beads are magnetized by electromagnet.

9. A device according to claim 1, further comprising a magnet that imparts magnetic property to the magnetic beads.

10. A device according to claim 1, further comprising a labeling chamber.

11. A device according to claim 1, further comprising a releasing chamber.

12. A device according to claim 1, further comprising a buffer inlet port.

13. A device according to claim 1, further comprising a waste outlet port.

14. A device according to claim 1, further comprising a detection module.

15. A device according to claim 14 wherein said detection module comprises:
a) a detection electrode;
b) a self-assembled monolayer;
c) a binding ligand;
d) a detection inlet port to receive said sample.

16. A device according to claim 1, further comprising a reagent storage well.

17. A device according to claim 1, further comprising a cell handling well.

18. A device according to claim 1, further comprising a reaction module.

19. A device according to claim 1, further comprising a separation module.

20. A device according to claim 1 further comprising a pump.

21. A device according to claim 1 further comprising a valve.

22. A microfluidic device comprising a solid support comprising:
a) a sample inlet port;
b) at least one microchannel comprising a gradient inducing feature coated with a magnetic material; and
c) a sample outlet port.

23. A device according to claim 22 wherein said microchannel comprises a plurality of gradient inducing features.

24. A device according to claim 22 wherein said gradient inducing feature is a sawtooth ridge.

25. A device according to claim 22 wherein said gradient inducing feature is a dome.

26. A device according to claim 22 wherein said gradient inducing feature has a diameter of between 1 μm and 1000 μm.

27. A device according to claim 22 wherein said magnetic material is an iron-nickel alloy.

28. A microfluidic device comprising a solid support comprising:
a) a sample inlet port;
b) at least one microchannel comprising at least one section filled with magnetic beads;
c) a sample outlet port; and
d) a detection module comprising:
i) a detection electrode;
ii) a self-assembled monolayer;
iii) a binding ligand; and
iv) a detection inlet port to receive said sample.

29. A method to process a target analyte in a sample comprising:
a) provide said target analyte labeled with a magnetic label; and
b) introducing said labeled target analyte to a microfluidic device comprising a solid support comprising:
i) a sample inlet port;
ii) at least one microchannel comprising at least one section with walls comprising magnetic beads;
iii) a sample outlet port;
under conditions whereby said labeled target analyte binds to said walls.

30. A method according to claim 29, further comprising:
a) washing away other components of said sample from said microchannel.

31. A method according to claim 29 or claim 30, further comprising treating the target analyte inside the channel.

32. A method according to claim 29 or claim 30, further comprising detecting the target analyte inside the magnetic microchannel.

33. A method according to any one of claims 29-31, further comprising eluting the target analyte or the analysis product from said walls.

34. A method according to claim 33, wherein said elution is achieved by reversing the electromagnet.

35. A method according to claim 33, wherein said elution is achieved by ferrofluid.

36. A method according to claim 33, wherein the elution is achieved by chemical disruption.

37. A method according to claim 33, wherein the elution is achieved by thermal disruption.

38. A method according to claim 29 wherein said target analyte is nucleic acid.

39. A method according to claim 29 wherein said target analyte is protein.

40. A method according to claim 29 wherein said target analyte is cell.

41. A method according to claim 29 wherein said target analyte is labeled in a labeling chamber.

42. A method according to claim 29, wherein said target analyte is further treated in a post-treatment module.

43. A method to process a target analyte in a sample comprising:
a) providing said target analyte labeled with a magnetic label; and
b) introducing said labeled target analyte to a microfluidic device comprising a solid support comprising:
i) a sample inlet port;
ii) at least one microchannel comprising a gradient inducing feature coated with a magnetic material; and
iii) a sample outlet port;
under conditions whereby said labeled target analyte is transported toward said gradient inducing feature.

44. A method to process a target analyte in a sample comprising:
a) provide said target analyte labeled with a magnetic label; and
b) introducing said labeled target analyte to a microfluidic device comprising a solid support comprising:
i) a sample inlet port;
ii) at least one microchannel comprising at least one section filled with magnetic beads;
iii) a sample outlet port; and
iv) a detection module comprising:
1) a detection electrode
2) a self-assembled monolayer;
3) a binding ligand; and
4) a detection inlet port to receive said sample.
under conditions whereby said labeled target analyte binds to said channel.
